(19) United States
(12) Patent Application Publication
(10) Pub. No.: US 2008/0124728 A1
(43) Pub. Date: May 29, 2008

(54) Removal of Molecular Assay Interferences for Nucleic Acids Employing Buffered Solutions of Chaotropes

(76) Inventor: Tony Baker, Sonora, CA (US)

Correspondence Address: BAKER BOTTS L.L.P.;1500 San Jacinto Plaza, 98 San Jacinto Blvd., Austin, TX, 78701, US

(21) Appl. No.: 11/854,330

(22) Filed: Sep. 12, 2007

Related U.S. Application Data
(60) Provisional application No. 60/825,379, filed on Sep. 12, 2006.

(51) Int. Cl. C12Q 1/68; C07H 21/00; C12N 9/16; C07K 14/805; C07K 14/47

(52) U.S. Cl. 435/6; 536/23.1; 435/196; 530/400

(57) ABSTRACT

The present disclosure relates to methods, compositions, and systems for reducing and/or eliminating (“suppressing”) undesirable effects of a masking agent on a molecular assay. In addition, the present disclosure relates to molecular assays of nucleic acids in bodily fluids and/or excretions. Suppressing undesirable effects of a masking agent may include, according to some embodiments, contacting a test sample with a composition comprising a chelator, a chelator enhancing component, and a buffer. A buffer, in some embodiments, may increase the concentration of chelators and/or chelator enhancing components that may be used without undesirable effects on a nucleic acid of interest (e.g., the integrity of the nucleic acid). In some embodiments, a buffer may enhance suppression of interference from masking agents. The amounts of the chelator(s) and the chelator enhancing component(s) may be selected such that interference of a masking agent on a molecular assay of a nucleic acid-containing test sample are suppressed.

### CROSS-REFERENCE TO RELATED APPLICATIONS

This application claims the benefit of U.S. Provisional Application Ser. No. 60/825,379, filed Sep. 12, 2006, entitled “Removal Of Molecular Assay Interferences For Nucleic Acids Employing Buffered Solutions Of Chaotropes.” This application is also related to U.S. patent application Ser. No. 09/932,122, filed Aug. 16, 2001, entitled “Removal of Molecular Assay Interferences,” by Tony Baker, which in turn was a continuation-in-part of co-pending application Ser. No. 09/805,785, filed Mar. 13, 2001, which is a continuation of application Ser. No. 09/185,402, filed Nov. 3, 1998, which is a continuation-in-part of application Ser. No. 08/988,029, filed Dec. 10, 1997. The entire contents of all the aforementioned applications are incorporated herein by reference.

### BACKGROUND

The present disclosure relates to compositions, methods, and systems for removing interferences from test samples, e.g., nucleic acid-containing samples obtained from living subjects, when they are submitted for or subjected to molecular assays.

The copying and cloning of virtually any nucleic acid sequence has been greatly facilitated by the polymerase chain reaction (PCR), which has become a fundamental methodology in molecular biology. In its simplest form, PCR is an in vitro method for the enzymatic synthesis of specific DNA sequences. In brief, PCR may involve hybridizing primers to denatured strands of a target nucleic acid or template in the presence of a polymerase enzyme and nucleotides under appropriate reaction conditions. The polymerase enzyme (usually a thermostable DNA polymerase) then recognizes the primer hybridized to the template and processes a primer extension product complementary to the template. The resultant template and primer extension product may then be subjected to further rounds of subsequent denaturation, primer hybridization, and extension as many times as desired in order to increase (or amplify) the amount of nucleic acid which has the same sequence as the target nucleic acid. Commercial vendors market PCR reagents and publish PCR protocols. PCR may be capable of producing a selective enrichment of a specific DNA sequence by a factor of 10⁹. The method is described in, e.g., U.S. Pat. Nos. 4,683,202; 4,683,195; 4,800,159; and 4,965,188, and in Saiki et al., 1985, Science 230:1350, all of which are incorporated herein by this reference.

The optimal efficiency of the amplification reaction, however, may be compromised by a number of unwanted side reactions. For example, many PCR procedures yield non-specific by-products caused by mispriming of the primers and template. Primers hybridizing to each other may also result in lost efficiency. This problem may be particularly acute when the target nucleic acid is present in very low concentrations and may obscure any amplified target nucleic acid (i.e., may produce high background).

Also, masking agents which interfere or inhibit such molecular assays as PCR are a problem in the art. Such inhibitors, which include leukocyte esterases, heme proteins, e.g., myoglobin and hemoglobin analogues, oxidation and breakdown products such as ferritins, methemoglobin, sulfhemoglobin and bilirubin, affect the accuracy of the assay, masking the true or detectable amount of, e.g., DNA in the sample. It is also conceivable that, e.g., a human sample containing genetic material for analysis could be spiked or doped with such agents to render a molecular assay done on the sample less trustworthy, or inconclusive.

Modern testing and treatment procedures have successfully reduced the prevalence and severity of many infectious diseases. For example, sexually-transmitted disease (STD) clinics regularly screen and treat patients for such diseases as gonorrhea and syphilis. Infectious agents such as gonococci may be identified by analyzing a DNA sample. Genetic transformation tests (GTT), such as the Gonostat® procedure (Sierra Diagnostics, Inc., Sonora, Calif.), can be used to detect gonococcal DNA in specimens taken from the urethra of men, and the cervix and anus of women. See, e.g., Jaffe et al., Diagnosis of gonorrhea using a genetic transformation test on mailed clinical specimens, J. Inf. Dis. 1982; 146:275-279, and Whittington et al., Evaluation of the genetic transformation test. Abstr. Ann. Meeting. Am. Soc. Microbiol. 1983; p. 315. The Gonostat® assay is discussed in Zubrzycki et al., Laboratory diagnosis of gonorrhea by a simple transformation test with a temperature-sensitive mutant of Neisseria gonorrhoeae, Sex. Transm. Dis. 1980; 7:183-187. The Gonostat(3) GTT, for example, may be used to detect, e.g., gonococcal DNA in urine specimens. The Gonostat assay uses a test strain, Neisseria gonorrhoeae, ATCC 31953, which is a mutant strain that is unable to grow into visible colonies on chocolate agar at 37° C. in 5% CO₂. Gonococcal DNA extracted from clinical material can restore colony growth ability to this test strain.

Such tests may be used to detect DNA in such bodily fluids and excretions as urine, blood, blood serum, amniotic fluid, spinal fluid, conjunctival fluid, salivary fluid, vaginal fluid, stool, seminal fluid, and sweat. Another test that can be used to identify DNA in a bodily fluid is PCR, since it uses discrete nucleic acid sequences and therefore can be effective even in the absence of intact DNA.

Still other methods exist that can amplify or detect specific nucleic acid sequences such as DNA or RNA. These methods include, but are not limited to, the ligase amplification reaction (LCR), hybridization, RT-PCR, NASBA, SDA, LCx, and genetic transformation testing. However, these methods are also vulnerable to interference by masking agents.

### SUMMARY

Therefore, there continues to be a need for improved methods of isolation and preservation of nucleic acids, including DNA and RNA, such that these nucleic acids can be used in procedures for analysis, detection, and amplification while minimizing the effects of masking agents described above.

The present disclosure relates, in some embodiments, to compositions, systems, and methods for preserving nucleic acids and/or preventing interference from masking agents in assays such as PCR. For example, in some embodiments, a solution may include a chaotropic agent and a buffer, in which the concentration of the chaotropic agent may be up to about 9 M.

The present disclosure relates, in some embodiments, to compositions, systems, and methods for assaying nucleic acids in bodily samples, e.g., fluids and excretions such as urine and blood. Without limiting any embodiment to a particular theory or view, some compositions, systems, and/or method may remove and/or inactivate one or more masking agents (e.g., methemoglobin), such that they no longer interfere with the accuracy or sensitivity of the molecular assay. Compositions, systems, and methods according to some embodiments have been found to also surprisingly increase the signal obtained with nucleic acid testing methods such as the polymerase chain reaction, LCx, (Abbott Laboratories) and genetic transformation testing. In some embodiments of the disclosure, hybridization in molecular assays such as nucleic acid testing methods may be improved, compared to when such assays are carried out without employing an embodiment of the present disclosure.

In some embodiments, the disclosure relates to methods of suppressing the action of masking agents of molecular assays, with the result being that the assay may be carried out at a much higher confidence level. The masking agents that are present in a nucleic acid-containing test sample may be suppressed by contacting the test sample with an amount of one or more divalent metal chelators (e.g., ethylenediaminetetraacetic acid, 1,2-bis(2-aminophenoxy)ethane-N,N,N′,N′-tetraacetic acid, and/or salts thereof) and an amount of one or more chelator enhancing components (e.g., lithium chloride, guanidinium chloride, guanidinium thiocyanate, guanidinium isothiocyanate, sodium perchlorate, and/or sodium salicylate) in a buffered solution. The concentrations of the divalent metal chelator(s) and the chelator enhancing component(s) may be selected such that the masking agents are suppressed, and upon contact with the divalent metal chelator(s)/chelator enhancing component(s), the masking agents are suppressed. The concentration of a divalent metal chelator may be from about 0.001 M to about 0.1 M, and the concentration of a chelator enhancing component (e.g., a chaotrope) may be from about 0.1 M to 9 M. Exact concentrations of a chelator enhancing component may be determined by one of ordinary skill in the art having the benefit of the present disclosure depending upon the particular chelator enhancing component or components used, the quantity of nucleic acid in the solution, and/or the quantity and type of masking agents that are or are expected to be present. The concentration of a chelator enhancing component may be at least about 1 M, and a divalent metal chelator may be present in a concentration of at least about 0.01 M. The buffer may be present in sufficient concentration to result in a pH from about 4.5 to about 8.0. Suitable buffers may include HEPES, potassium acetate, sodium phosphate, and/or tris(hydroxyamino)methane (Tris). Other buffers may alternatively be used. Additionally, the solution used to contact the test sample may include one or more nonionic detergents such as Tween 20.

In some embodiments, the disclosure relate to methods of improving the signal response of a molecular assay. Masking agents in a nucleic acid-containing test sample may be suppressed, for example, by contacting the test sample with an amount of one or more divalent metal chelator(s) and an amount of one or more chelator enhancing components in a buffered solution. The concentrations of the divalent metal chelator(s) and chelator enhancing component(s) may be selected such that the masking agents are suppressed. Molecular analytes of interest from the preserved test sample may be extracted; and a molecular assay may be conducted on the extracted molecular analytes of interest, whereupon the signal response of the molecular assay is improved. Signal response may be enhanced, in part, due to enhanced hybridization as a result of the use of the reagents of the present invention.

The disclosure, according to some embodiments, relates to methods of improving hybridization of nucleic acids, including contacting a test nucleic acid with a reagent comprising an amount of at least one divalent metal chelator (e.g., in the concentration range of from about 0.001 M to 0.1 M) and an amount of at least one chelator enhancing component (e.g., in the concentration range of from about 0.1 M to 9 M), such that a test solution is formed; and contacting the test solution with a target nucleic acid under conditions that permit hybridization.

Compositions, systems, and methods of the disclosure may further include an amount of at least one enzyme-inactivating component such as manganese chloride, sodium lauroyl sarcosinate (Sarkosyl) and/or sodium dodecyl sulfate, at a concentration of, for example, up to about 5% (w/v).

Accordingly, the disclosure provides a method for amplifying target nucleic acids, comprising contacting a target nucleic acid with a solution comprising a chelator, a chelator enhancing component, and a buffer under conditions which allow for an amplification reaction to occur. The disclosure may also be useful in commercial applications including specialty chemicals and instrumentation for utilizing this technology, e.g., probe-based diagnostics, microarray/DNA Chip methods, PCR (e.g., hot-start PCR) hybridization and amplification, SNP analysis, and/or DNA sequencing. Other applications may include drug discovery and the study of drug response genes (pharmacogenomics), drug delivery and therapeutics.

In some embodiments manipulation of the reaction mixture may not be required following initial preparation. Thus, some embodiments of the disclosure may be used in existing automated PCR amplification systems and/or with in situ amplification methods where the addition of reagents after the initial denaturation step is inconvenient and/or impractical.

### BRIEF DESCRIPTION OF THE DRAWINGS

Some embodiments of the disclosure may be understood with reference to the specification, appended claims, and accompanying drawings, wherein:

FIG. 1 is a graph of DNA concentration in urine according to prior art.

FIG. 2 is a graph of eight day serial data on urine according to prior art;

FIG. 3 is a graph of DNA concentration in serum according to prior art;

FIG. 4 is a graph showing the interference of methemoglobin on PCR absorbance in a PCR amplification assay on hepatitis B sequences MD03/06 in untreated serum;

FIG. 5 is a graph showing the improvement in attenuating the interference of methemoglobin on PCR absorbance in a PCR amplification assay on hepatitis B sequences MD03/06 in serum which has been treated with a composition of the disclosure.

FIG. 6A illustrates the synergistic effect provided by the components of some specific example embodiments of the disclosure in protecting hepatitis B sequences in serum stored at room temperature and subsequently subjected to MD03/06 PCR detection.

FIG. 6B illustrates the synergistic effect provided by the components of some specific example embodiments of the disclosure in protecting hepatitis B sequences in serum stored at room temperature and subsequently subjected to MD03/06 PCR detection.

FIG. 6C illustrates the synergistic effect provided by the components of some specific example embodiments of the disclosure in protecting hepatitis B sequences in serum stored at room temperature and subsequently subjected to MD03/06 PCR detection.

FIG. 6D illustrates the synergistic effect provided by the components of some specific example embodiments of the disclosure in protecting hepatitis B sequences in serum stored at room temperature and subsequently subjected to MD03/06 PCR detection.

FIG. 6E illustrates the synergistic effect provided by the components of some specific example embodiments of the disclosure in protecting hepatitis B sequences in serum stored at room temperature and subsequently subjected to MD03/06 PCR detection.

FIG. 6F illustrates the synergistic effect provided by the components of some specific example embodiments of the disclosure in protecting hepatitis B sequences in serum stored at room temperature and subsequently subjected to MD03/06 PCR detection.

FIG. 7 graphically illustrates a comparison of signal response in PCR assays wherein the DNA has been treated with a specific example embodiment of the disclosure, and one which has not.

FIG. 8 illustrates the efficacy of some specific example embodiments of the present disclosure to enhance signal response of a branched DNA assay of blood plasma samples subjected to various storage conditions.

FIG. 9 illustrates the efficacy of some specific example embodiments of the present disclosure to enhance signal response of a branched DNA assay of blood serum and plasma samples.

FIG. 10 is a graph showing the effect of buffered solutions with high concentrations of chaotropes versus non-buffered solutions with equivalent concentrations of chaotropes in protecting 100 copies of MOMP chlamydia target DNA in fresh urine at 30° C.

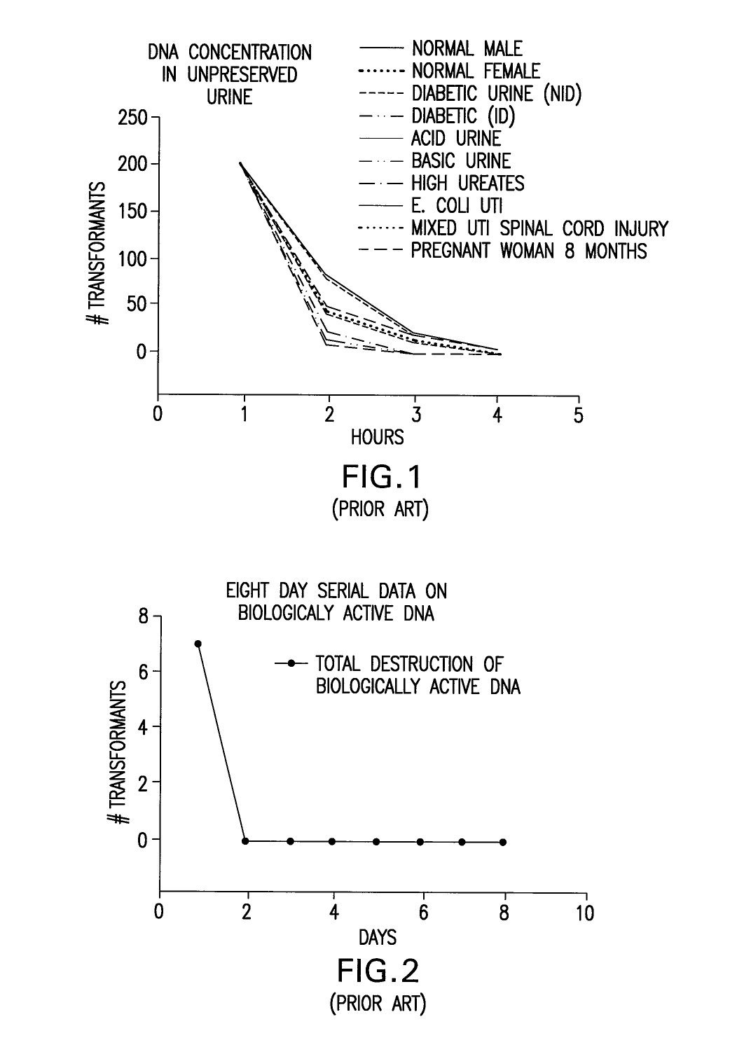

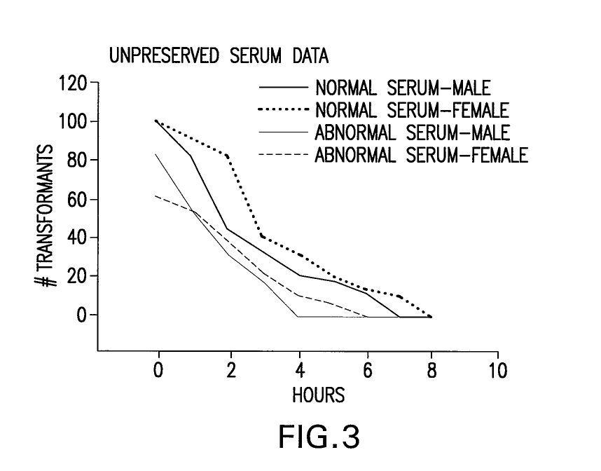

### DETAILED DESCRIPTION

The present disclosure relates to methods, compositions, and systems for reducing and/or eliminating (“suppressing”) undesirable effects of a masking agent on a molecular assay. In addition, the present disclosure relates to molecular assays of nucleic acids in bodily fluids and excretions, such as urine, blood, blood serum, amniotic fluid, spinal fluid, conjunctival fluid, salivary fluid, vaginal fluid, stool, seminal fluid, and sweat. Interference that may be caused by masking agents may be suppressed, according to some embodiments, by contacting a test sample with an amount of one or more chelators (e.g., divalent metal chelators) and an amount of one or more chelator enhancing components in a buffered solution. A buffer may, in some embodiments, increase the concentration of chelators and/or chelator enhancing components that may be used without undesirable effects on a nucleic acid of interest (e.g., the integrity of the nucleic acid). In some embodiments, a buffer may enhance suppression of interference from masking agents. The amounts of the chelator(s) and the chelator enhancing component(s) may be selected such that interference of a masking agent on a molecular assay of a nucleic acid-containing test sample are suppressed.

The term “molecular assay” as used herein may be an assay or technique that involves sequence-specific interactions between a nucleic acid and either another nucleic acid or a protein molecule. The assay may involve additional steps that may occur following sequence-specific interactions. “Molecular assay” may include nucleic acid amplification techniques such as PCR; RT-PCR (e.g., U.S. Pat. No. 4,683,202); LCR (ligase chain reaction) described in, e.g., EP-A-0320308; the “NASBA” or “3SR” technique described in, e.g., Proc. Natl. Acad. Sci. Vol. 87 pp. 1874-1878 March 1990 and Nature Vol. 350, No. 634. PP 91-92 Mar. 7, 1991; the “SDA” method described in, e.g., Nucleic Acid Research, Vol. 20 PP 1691-1696; LCx; hybridization; and genetic transformation testing (GTT).

The term “masking agent” as used herein may be a compound that inhibits sequence-specific interactions of any molecular assay, as defined above, other than by competitive inhibition. The term “interferent(s) of molecular assay(s)” is used synonymously with “masking agents.” “Masking agents” and/or “interferents of molecular assay(s)” may include compounds which interfere or otherwise reduce the accuracy of the assay, masking the true or detectable amount of the nucleic acid in the sample. Examples are leukocyte esterases, heme proteins, myoglobin and hemoglobin analogs, derivatives, oxidation and breakdown products such as ferritins, methemoglobin, sulfhemoglobin and bilirubin.

“Metal cations” may include cations associated with metal-dependent enzymes. Examples of metal cations include cations of iron, aluminum, copper, cobalt, nickel, zinc, cadmium, magnesium, and calcium. Metal cations of particular interest include magnesium (e.g., Mg⁺²) and calcium (e.g., Ca⁺²).

The term “bodily fluid” as used herein may be and/or may comprise any fluid originating from an organism upon which a molecular assay may be performed. The term “bodily fluid” may include, e.g., urine, blood, blood serum, amniotic fluid; cerebrospinal fluid, spinal fluid; synovial fluid, conjunctival fluid, salivary fluid, vaginal fluid, stool, seminal fluid, lymph, bile, tears, and/or sweat.

“Sample” may include a composition that is to be tested for the presence of a nucleic acid, protein or other macromolecule of interest (quantitatively and/or quantitatively) and/or cell of interest. A sample may include a sample of tissue or fluid isolated from an individual or individuals, including bodily fluids, skin, blood cells, organs, tumors, and also to samples of in vitro cell culture constituents (including but not limited to conditioned medium resulting from the growth of cells in cell culture medium, recombinant cells and cell components).

“Divalent metal chelator” may include compounds which chelate and/or remove divalent metal cations. In some embodiments, metal dependent enzymes such as deoxyribonucleases may be inactivated in the presence of one or more chelators. Deoxyribonucleases, for example, have been found to degrade gonococcal DNA in urine over time. Examples of chelators (e.g., divalent metal chelators) may include ethylenediaminetetraacetic acid (EDTA); imidazole; [ethylenebis(oxyethylenenitrilo)]tetraacetic acid (EGTA); iminodiacetate (IDA); 1,2-bis(2-aminophenoxy)ethane-N,N,N′,N′-tetraacetic acid (BAPTA); bis(5-amidino-2-benzimidazolyl)methane (BABIM) or salts thereof. For example, divalent metal chelators may include EDTA, EGTA and/or BAPTA. The concentration of a chelator (e.g., a divalent metal chelator) in the final reaction solution including the nucleic acid may be from about 0.001 M to about 0.6 M. A final reaction solution including a nucleic acid may also be referred to herein as a “test sample.” The concentration of a chelator (e.g., a divalent metal chelator) in the final reaction solution including the nucleic acid according to some embodiments, may be from about 0.1 M to about 0.5 M. In some embodiments, the concentration of a chelator (e.g., a divalent metal chelator) in the final reaction solution including a nucleic acid may from about 0.2 M to about 0.4 M. A final reaction solution including a nucleic acid may be prepared by mixing a sample including the nucleic acid with a concentrated reagent stock solution (e.g., in a ratio of 9:1), so that the concentration of the divalent metal chelator in the concentrated reagent stock solution is from about 0.01 M to about 6.0 M. The concentration of a divalent metal chelator in a concentrated reagent stock solution may be from about 1.0 M to about 5.0 M and/or from about 2.0 M to about 4.0 M.

‘Chelator enhancing component’ may include compounds which, for example, assist a divalent metal chelator in protecting nucleic acids in a bodily fluid. In some embodiments, a chelator enhancing component may inactivate one or more metal independent enzymes that may be found in a sample. A metal independent enzyme may comprise a DNA ligase, e.g., T4 DNA ligase; a DNA polymerase such as a T7 DNA polymerase; exonucleases such as a 3′exonuclease, exonuclease-2, and/or a 5′ exonuclease; a kinase such as T4 polynucleotide kinase; a phosphatase such as BAP and/or CIP phosphatase; a nuclease such as BL31 nuclease and/or XO nuclease; and a RNA-modifying enzyme such as Escherichia coli RNA polymerase, a SP6 RNA polymerase, a T7 RNA polymerase, a T3 RNA polymerase, and/or a T4 RNA ligase. Lithium chloride, guanidinium chloride, guanidinium thiocyanate, guanidinium isothiocyanate, sodium salicylate, sodium perchlorate, sodium thiocyanate, and/or sodium isothiocyanate have been found to be effective. A chelator enhancing component may be a chaotrope and/or may disrupt secondary, tertiary, and/or quaternary structure of a metal dependent enzyme. The concentration of a chelator enhancing component in the final reaction solution including the nucleic acid may be from about 0.01 M to about 0.9 M. For example, the concentration of a chelator enhancing component in the final reaction solution including the nucleic acid may be from about 0.1 M to about 0.8 M and/or from about 0.2 M to about 0.7 M. As indicated above, the final reaction solution including the nucleic acid may be prepared by mixing a sample including a nucleic acid with a concentrated reagent stock solution (e.g., in a ratio of 9:1). Typically, the concentration of a chelator enhancing component in a concentrated reagent stock solution may be from about 0.1 M to about 9 M and/or from about 2 M to about 7 M.

The term “buffer” and variants thereof such as “buffered solution” may comprise a base and its conjugate acid present in a solution in a quantity sufficient to maintain a desired pH value. Suitable buffers and buffer concentrations are described further in detail below.

“Nucleic acid”, “polynucleotide” and “oligonucleotide” may include DNA molecules (e.g., cDNA or genomic DNA), RNA molecules (e.g., mRNA), analogues of the DNA or RNA generated using nucleotide analogues or using nucleic acid chemistry, and PNA (protein nucleic acids); modified nucleotides such as methylated or biotinylated nucleotides, primers, probes, oligomer fragments, oligomer controls and unlabeled blocking oligomers; polydeoxyribonucleotides (containing 2-deoxy-D-ribose), polyribonucleotides (containing D-ribose), and/or any other type of polynucleotide which is an N-glycoside of a purine or pyrimidine base, or modified purine or pyrimidine base. There is no intended distinction in length between the term “nucleic acid,” “polynucleotide,” and “oligonucleotide,” and these terms will be used interchangeably. These terms may refer only to the primary structure of the molecule. Thus, these terms include double- and single-stranded DNA, as well as double- and single-stranded RNA. Oligonucleotides may include a sequence of approximately at least about 6 nucleotides, at least about 10-12 nucleotides, and/or at least about 15-20 nucleotides corresponding to a region of the designated nucleotide sequence.

Oligonucleotides are not necessarily physically derived from any existing or natural sequence but may be generated in any manner, including chemical synthesis, DNA replication, reverse transcription or a combination thereof. Oligonucleotides and/or nucleic acids may include those which, by virtue of its origin or manipulation: (1) are not associated with all or a portion of the polynucleotide with which it is associated in nature; and/or (2) are linked to a polynucleotide other than that to which it is linked in nature; and/or (3) are not found in nature.

“Corresponding” means identical to or complementary to a designated sequence.

“Primer” or “nucleic acid primer” may refer to more than one primer and may include oligonucleotides, whether occurring naturally, as in a purified restriction digest, or produced synthetically, which are capable of acting as a point of initiation of synthesis along a complementary strand when placed under conditions in which synthesis of a primer extension product which is complementary to a nucleic acid strand is catalyzed. Primers may be from about 10 to about 100 bases and are designed to hybridize with a corresponding template nucleic acid. Primer molecules may be complementary to either the sense or the anti-sense strand of a template nucleic acid and/or may be used as complementary pairs that flank a nucleic acid region of interest. Synthesis conditions may include the presence of four different deoxyribonucleoside triphosphates and a polymerization-inducing agent such as DNA polymerase or reverse transcriptase, in a suitable reaction mixture (“reaction mixture” includes substituents which are cofactors, or which affect pH, ionic strength, or other parameters affecting the efficiency of the reaction), and at a suitable temperature. A primer may be single-stranded for maximum efficiency in amplification.

The “complement” of a nucleic acid sequence may include, for example, oligonucleotides which, when aligned with the nucleic acid sequence such that the 5′ end of one sequence is paired with the 3′ end of the other, are in “antiparallel association.” Certain bases not commonly found in natural nucleic acids may be included, for example, inosine and/or 7-deazaguanine. Complementarity need not be perfect; stable duplexes may contain mismatched base pairs or unmatched bases. One of ordinary skill in the art having the benefit of the present disclosure may determine duplex stability empirically considering a number of variables including, for example, the length of the oligonucleotide, base composition and/or sequence of the oligonucleotide, ionic strength, and/or incidence of mismatched base pairs.

“Target sequence” or “target nucleic acid sequence” may refer to a region of the oligonucleotide which is to be either amplified, detected or both. When amplification is intended, the target sequence resides between the two primer sequences used for amplification.

“Probe” may refer to a labeled oligonucleotide which forms a duplex structure with a sequence in a target nucleic acid, due to, for example, complementarity of at least one sequence in the probe with a sequence in the target region. A probe may not contain a sequence complementary to sequence(s) used to prime a polymerase chain reaction. Generally the 3′ terminus of a probe may be blocked to prohibit incorporation of the probe into a primer extension product. Blocking may be achieved, for example, by using non-complementary bases and/or by adding a chemical moiety such as biotin or a phosphate group to the 3′ hydroxyl of the last nucleotide, which may, depending upon the selected moiety, serve a dual purpose by also acting as a label for subsequent detection and/or capture of the nucleic acid attached to the label. Blocking may also be achieved, for example, by removing the 3′-OH and/or by using a nucleotide that lacks a 3′-OH such as a dideoxynucleotide.

“Polymerase” may include, for example, any one of, or a mixture of, the nucleotide polymerizing enzymes E. coli DNA polymerase I, Taq polymerase, Klenow fragment of E. coli DNA polymerase I, T4 DNA polymerase, reverse transcriptase where the template is RNA and the extension product is DNA, or a thermostable DNA polymerase.

“Thermostable nucleic acid polymerase” may refer to an enzyme which is relatively stable to heat when compared, for example, to nucleotide polymerases from E. coli and which catalyzes the polymerization of nucleoside triphosphates. Generally, a thermostable nucleic acid polymerese may initiate synthesis at the 3′-end of the primer annealed to the target sequence, and will proceed in the 5′-direction along the template, and if possessing a 5′-to-3′ nuclease activity, hydrolyzing intervening, annealed probe to release both labeled and unlabeled probe fragments, until synthesis terminates. A thermostable nucleic acid polymerese may include, for example, a thermostable enzyme isolated from Thermus aquaticus (Taq) described in U.S. Pat. No. 4,889,818. A method for using this polymerese in conventional PCR is described in, e.g., Saiki et al., 1988, Science 239:487, both incorporated herein by this reference. Taq DNA polymerase may have a DNA synthesis-dependent, strand replacement 5′-3′ exonuclease activity (see Gelfand, “Taq DNA Polymerase” in PCR Technology: Principles and Applications for DNA Amplification, Erlich, Ed., Stockton Press, N.Y. (1989), Chapter 2). Additional examples of thermostable nucleic acid polymerases may include polymerases extracted from the thermostable bacteria Thermus flavus, Thermus ruber, Thermus thermophilus, Bacillus stearothermophilus, Thermus lacteus, Thermus rubens, Thermotoga maritima, Thermococcus litoralls, Methanothermus fervidus, Thermus filiformis, Pyrococcus furiosus, a Thermotoga species, or a recombinant form thereof.

“Thermal cycle” may include any change in the incubation temperature of a nucleic acid sample designed to change the activity of a component of the sample such as, e.g., the binding affinity of a primer for a nucleic acid.

The terms “hybridize” and/or “hybridization” may include hydrogen bonding of complementary DNA and/or RNA sequences to form a duplex molecule. Hybridization may take place between a primer and template and/or between primers. Reactions between, when undesired or unintended, may be inhibited by using embodiments of compositions, systems, and/or methods of the disclosure.

The terms “amplification” and/or “amplify” may include reactions necessary to increase the number of copies of a nucleic acid sequence, such as a DNA sequence. For example, amplification may refer to the in vitro exponential increase in copy number of a target nucleic acid sequence, such as that mediated by a polymerase amplification reaction (e.g., PCR reaction). Other amplification reactions may include RT-PCR (see, e.g., U.S. Pat. No. 4,683,202; Mullis et al.), and a ligase chain reaction (Barany, Proc. Natl. Acad. Sci. USA 88:189-193 (1991)).

“Selective amplification” may refer to the preferential copying of a target or template nucleic acid of interest using a polymerase amplification reaction, such as PCR reaction. In a PCR reaction, this may be accomplished by the use of specific primers to delimit the sequence being copied.

Some embodiments of the disclosure may be practiced using one or more, conventional techniques of molecular biology, microbiology and/or recombinant DNA techniques, which are within the skill of those in the art. Such techniques are explained fully in the literature. See, e.g., Sambrook, Fritsch & Maniatis, Molecular Cloning: A Laboratory Manual, Second Edition (1989); Oligonucleotide Synthesis (M. J. Gait, ed., 1984); Nucleic Acid Hybridization (B. D. Hames & S. J. Higgins, eds., 1984); A Practical Guide to Molecular Cloning (B. Perbal, 1984); and a series, Methods in Enzymology (Academic Press, Inc.).

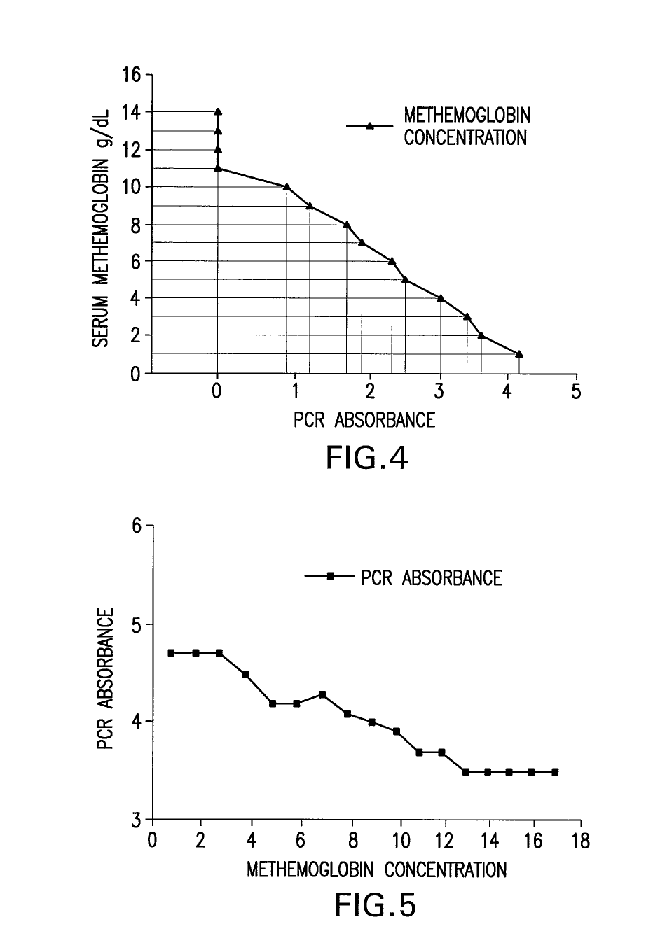

Some specific examples of embodiments of compositions of the disclosure have surprisingly been found to abate and/or remove the interference of masking agents, e.g., heme proteins including methemoglobin on PCR assays run on blood serum. FIGS. 4 and 5 illustrate examples of the improvement obtained by use of specific example embodiments disclosed herein. Increasing amounts of methemoglobin were spiked into untreated fresh human serum, to a concentration of 10 dl/ml. Serial PCR assays were run over a four hour period.

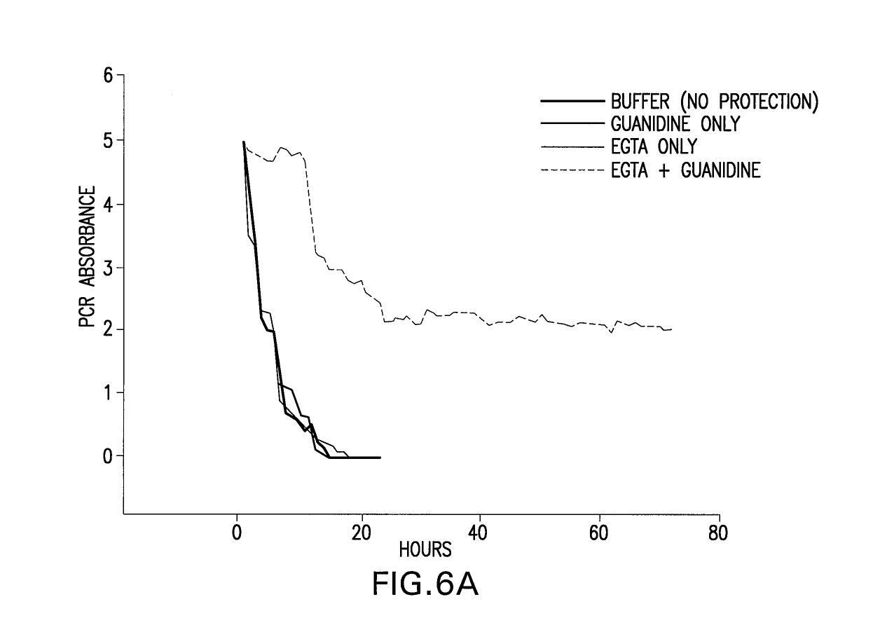

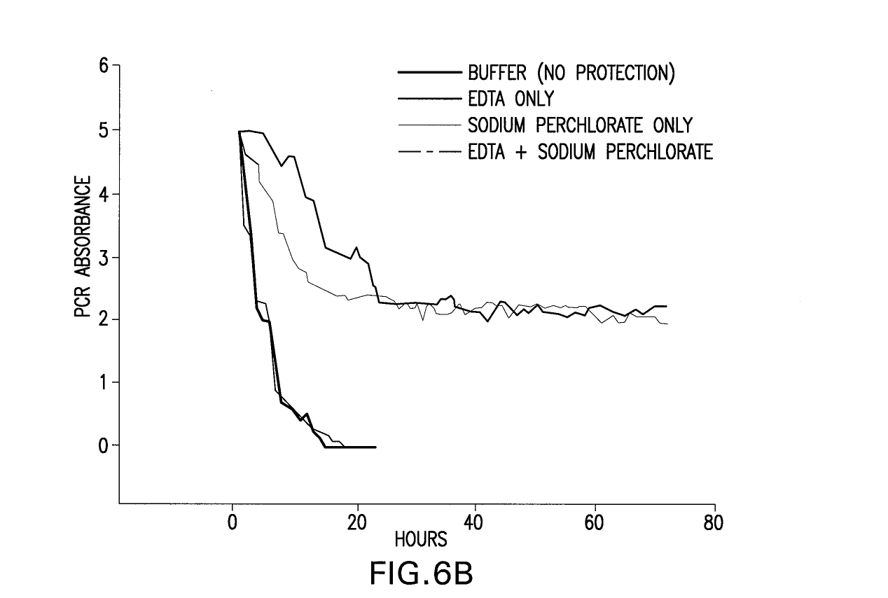

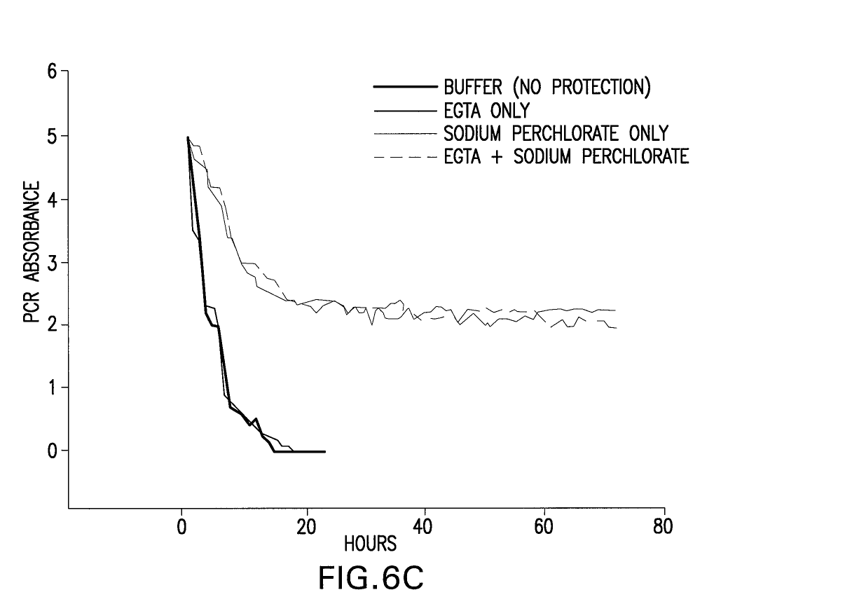

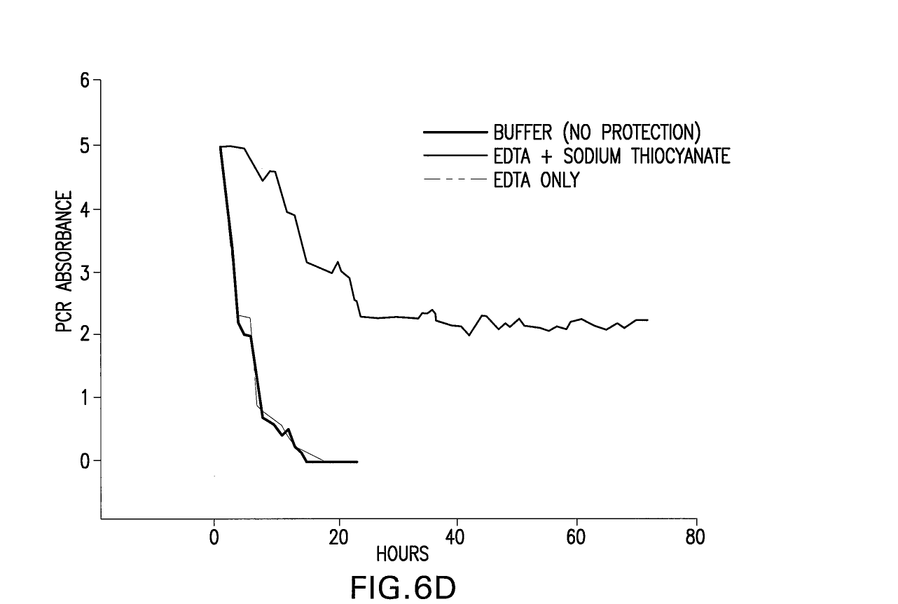

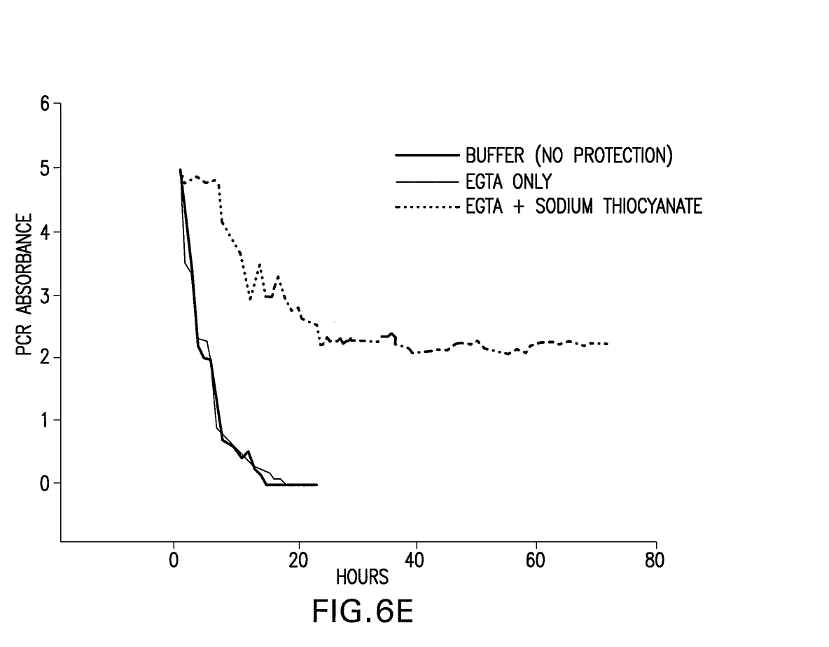

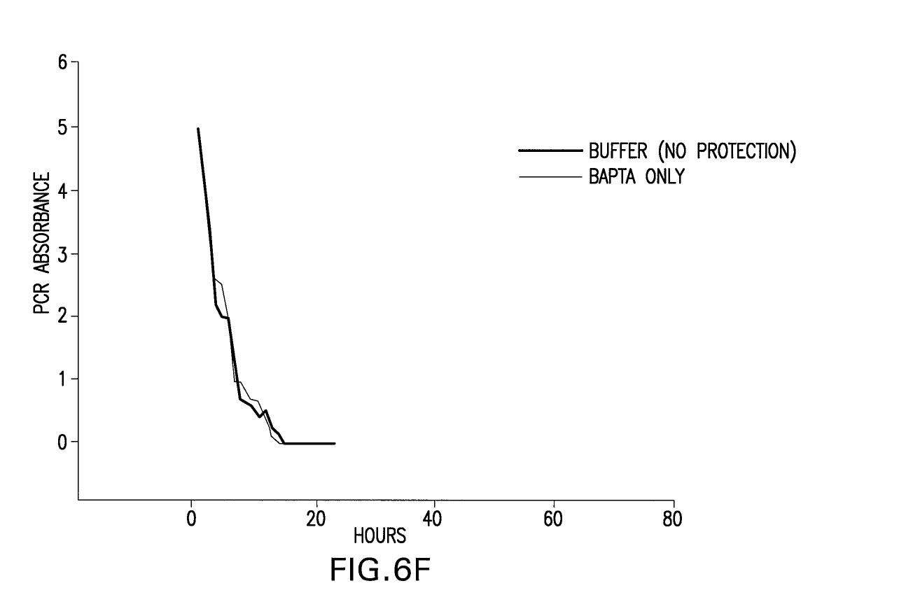

FIGS. 6A-6F illustrate an example of the surprising and synergistic effect obtained by the combination of divalent metal chelators and chelator enhancing components (i.e., 1 M sodium perchlorate/0.01 M EGTA) in protecting hepatitis B sequences in serum stored at room temperature and subsequently subjected to MD03/06 PCR detection. The protocol run was as above (i.e., as illustrated in FIGS. 6A-6F). It can be seen from the figures that compared to the addition of EGTA or sodium perchlorate individually, protection of Hep B sequences is dramatically increased when reagent solutions of the present invention are used.

In some embodiments, the disclosure also provides compositions, systems, and methods for the molecular assay of nucleic acids in other bodily fluids and excretions. These assays may be carried out with greater sensitivity, according to some embodiments, because compositions, systems, and methods of the disclosure have been found to surprisingly increase the signal obtained with such molecular assays as PCR. Additionally, hybridization in such nucleic acid testing methods is unexpectedly improved.

Unexpectedly, significant protection of nucleic acids in samples, blocking of the effects of masking agents, and increase of signal in such molecular assays as PCR has been found to occur when divalent metal chelators and chelator enhancing components as described above are used in a buffered solution. According to some embodiments, a buffer that results in a pH in the range of from about 4.5 to about 8.0 may be used. The pH may be in the range from about 6.9 to about 7.6 in some embodiments. Examples of buffers may include potassium acetate, sodium acetate, potassium phosphate, sodium phosphate, tris(hydroxymethyl)aminomethane (Tris), and/or (N-(2-hydroxyethyl)piperazine-N′-(2-ethanesulfonic acid) (HEPES). Other buffers that provide buffering capacity in these pH ranges may be used in compositions, systems and methods according to the present invention, including, but not limited to, MOPS buffer (3-(N-morpholino)propanesulfonic acid), ACES (2-[(2-amino-2-oxoethyl)amino]ethanoesulfonic acid) buffer, ADA (N-(2-acetamido)2-iminodiacetic acid) buffer, AMPSO (3-[(1,1-dimethyl-2-hydroxyethyl)amino]-2-propanesulfonic acid) buffer, BES (N,N-bis(2-hydroxyethyl)-2-aminoethanesulfonic acid buffer, Bicine (N,N-bis(2-hydroxyethylglycine) buffer, Bis-Tris (bis-(2-hydroxyethyl)imino-tris(hydroxymethyl)methane buffer, CAPS (3-(cyclohexylamino)-1-propanesulfonic acid) buffer, CAPSO (3-(cyclohexylamino)-2-hydroxy-1-propanesulfonic acid) buffer, CHES (2-(N-cyclohexylamino)ethanesulfoni c acid) buffer, DIPSO (3-[N,N-bis(2-hydroxyethyl)amino]-2-hydroxy-propanesulfonic acid) buffer, HEPPS(N-(2-hydroxyethylpiperazine)-N′-(3-propanesulfonic acid) buffer, HEPPSO(N-(2-hydroxyethyl)piperazine-N′-(2-hydroxypropanesulfonic acid) buffer, MES (2-(N-morpholino)ethanesulfonic acid) buffer, triethanolamine buffer, imidazole buffer, glycine buffer, ethanolamine buffer, phosphate buffer, MOPSO (3-(N-morpholino)-2-hydroxypropanesulfonic acid) buffer, PIPES (piperazine-N,N′-bis(2-ethanesulfonic acid) buffer, POPSO (piperazine-N,N′-bis(2-hydroxypropaneulfonic acid) buffer; TAPS(N-tris[hydroxymethyl)methyl-3-aminopropanesulfonic acid) buffer, TAPSO (3-[N-tris(hydroxymethyl)methylamino]-2-hydroxy-propanesulfonic acid) buffer, TES (N-tris(hydroxymethyl)methyl-2-aminoethanesulfonic acid) buffer, tricine (N-tris(hydroxymethyl)methylglycine buffer), 2-amino-2-methyl-1,3-propanediol buffer, and/or 2-amino-2-methyl-1-propanol buffer. Particularly preferred buffer solutions, including their pH values and concentrations, as well as recipes for preparing the buffer solutions, are described in the Examples.

It has also unexpectedly been found that significant protection of nucleic acids in samples, blocking of the effects of masking agents, and/or an increase of signal in such molecular assays as PCR occur when a nonionic detergent is included in the buffered solution described above. An example of a nonionic detergent is a polyoxyethylene sorbitan monolaurate. Another example of a nonionic detergent is a polyoxyethylene (20) sorbitan monolaurate such as Tween 20. A concentration of a nonionic detergent (e.g. Tween 20) may be about 0.1% (w/v) in the concentrated reagent stock solution. This may correspond to a concentration of about 0.01% (w/v) in the test sample. Additional nonionic detergents are known in the art, including, but not limited to, octyl- and nonylphenoxypolyethoxylethanols (Nonidet detergents), octyl glucopyranosides, dodecyl maltopyranosides, heptyl thioglucopyranosides, Big CHAP detergents, Genapol X-80, Pluronic detergents, polyoxyethylene esters of alkylphenols (Triton), and/or derivatives and analogues of these detergents.

Compositions, systems, and methods of the disclosure may include an amount of at least one enzyme inactivating component (e.g., manganese chloride, sodium lauroyl sarcosinate (Sarkosyl), or sodium dodecyl sulfate). An enzyme inactivating component may be present at a concentration of up to about 5% (w/v) in the final reaction solution including the nucleic acid.

Compositions, systems, and/or methods of the disclosure may be used in some embodiments to preserve prokaryotic (e.g., gonococcal DNA), human, bacterial, fungal, and/or viral nucleic acids (e.g., DNA and/or RNA). Without limiting any particular embodiment to any specific mechanism or theory of action, the efficacy of one or more compositions, systems, and/or methods of the disclosure may be due, at least in part, to inactivation of one or more metal-dependent enzymes and/or metal independent enzymes, which may be present in bodily fluids such as blood or urine and which may be destructive to DNA integrity.

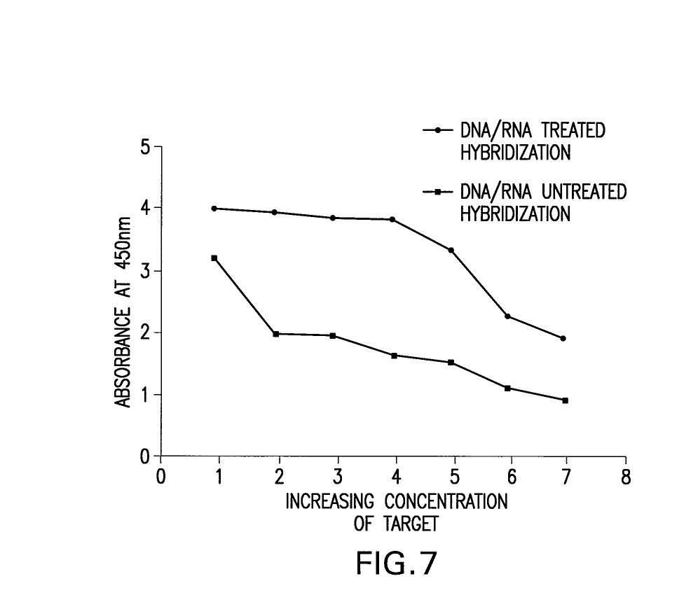

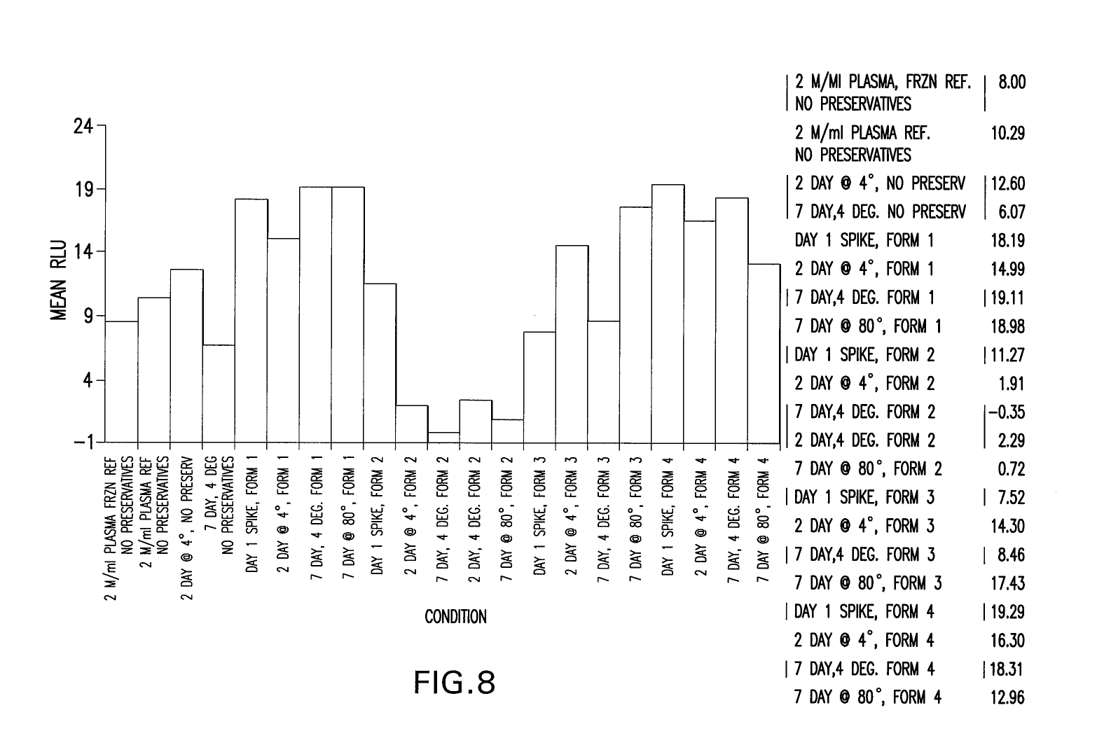

Compositions, systems, and methods of the disclosure, according to some embodiments, have been found to increase the signal obtained with such nucleic acid testing methods as the polymerase chain reaction (PCR), LCₓ, and genetic transformation testing (GTT). For example, some embodiments of the disclosure been found to surprisingly and unexpectedly enhance hybridization in such nucleic acid testing methods as PCR. FIGS. 7 and 8 illustrate an example of the improvement in hybridization obtained by use of a composition disclosed herein on the hybridization of penicillinase-producing Neisseria gonorrhoeae (PPNG) DNA and PPNG-C probe.

The disclosure relates, in some embodiments, to methods of improving hybridization of nucleic acids, including contacting a test nucleic acid with a nucleic acid reagent solution comprising (a) an amount of a divalent metal chelator in the range of, for example, about 0.001 M to 0.1 M (b) an amount of at least one chelator enhancing component as described above in the range of, for example, about 0.1 M to 9 M, (c) optionally, a buffer so that the solution is buffered, and, (d) optionally, a nonionic detergent as described above such that a test solution is formed; and contacting the test solution with a target nucleic acid under conditions that permit for hybridization, such that hybridization occurs.

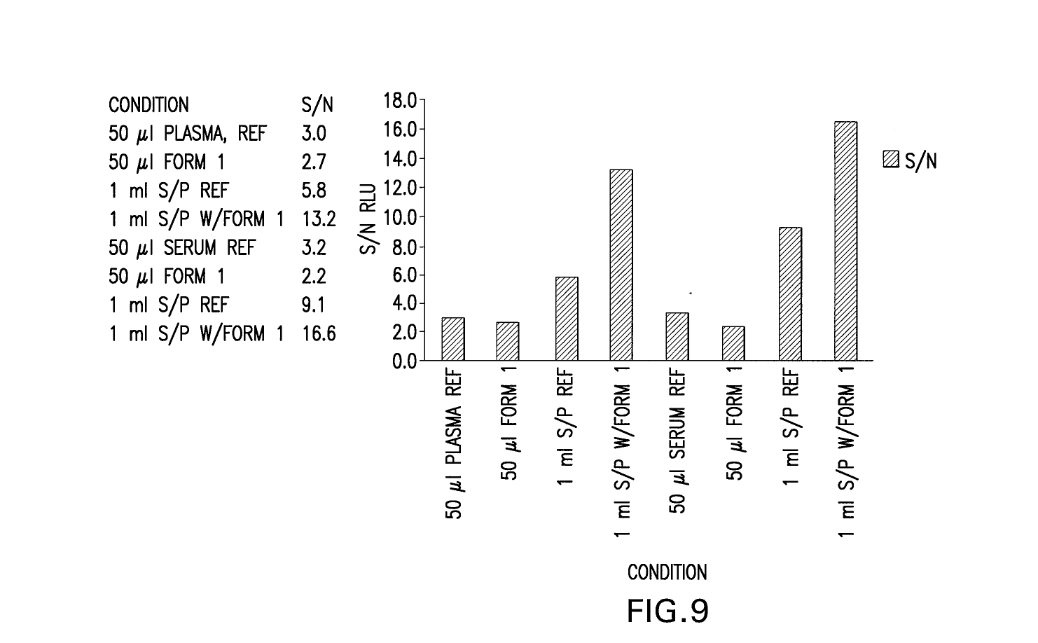

FIGS. 8 and 9 illustrate examples of the efficacy of some specific example embodiments of compositions, systems, and methods of the disclosure in improving the results obtained with a branched DNA (bDNA) assay (Chiron). In the tests run in FIG. 8, a bDNA assay was used to assess the effect of specific example embodiments of compositions of the disclosure. DNA sequences from hepatitis C virus were spiked into serum and plasma. The treated serum and plasma were mixed with 9 ml of serum or plasma and 1 ml of reagent. The following formulations were used: 1) 1 M guanidine HCl/0.01 M EDTA, 2) 1 M sodium perchlorate/0.01 M BAPTA, 3) 1 M sodium thiocyanate/0.01 M EGTA, and 4) 1 M lithium chloride/0.01 M EGTA. The formulations were stored for seven days at 4° C. The bDNA assay relies on hybridization. The more than doubling of the absorbance results indicates an enhancement of hybridization/annealing of the target sequences.

FIG. 9 illustrates an example of a serum v. plasma study. 50 ml samples of fresh human plasma, and 1 ml samples of fresh human serum were treated with 1M guanidine HCL/0.01M EDTA and the bDNA assay was run on these samples after the samples were stored at −6.7° C. (20° F.) for 48 hours. Results were compared to untreated samples. Again, the more than doubling of the absorbance results indicates an enhancement of hybridization/annealing of the target sequences.

Some embodiments of the disclosure may be conveniently incorporated into established protocols without the need for extensive re-optimization.

In some embodiments, PCR may be carried out as an automated process utilizing a thermostable enzyme. The reaction mixture may be cycled through a denaturing step, a probe and primer annealing step, and a synthesis step, whereby cleavage and displacement occurs simultaneously with primer-dependent template extension. A DNA thermal cycler, which is specifically designed for use with a thermostable enzyme, may be employed.

Detection and/or verification of the labeled oligonucleotide fragments may be accomplished by a variety of methods and may be dependent on the source of the label or labels employed. Reaction products, including the cleaved labeled fragments, may be subjected to size analysis. Methods for determining the size of the labeled nucleic acid fragments may include, for example, gel electrophoresis, sedimentation in gradients, gel exclusion chromatography and/or homochromatography.

During or after amplification, separation of the labeled fragments from the PCR mixture may be accomplished by, for example, contacting the PCR mixture with a solid phase extractant (SPE). For example, materials having an ability to bind oligonucleotides on the basis of size, charge, and/or interaction with the oligonucleotide bases can be added to the PCR mixture, under conditions where labeled, uncleaved oligonucleotides are bound and short, labeled fragments are not. Such SPE materials may include ion exchange resins or beads, such as the commercially available binding particles Nensorb (DuPont Chemical Co.), Nucleogen (The Nest Group), PEI, BakerBond.™. PEI, Amicon PAE 1000, Selectacel™, PEI, Boronate SPE with a 3′-ribose probe, SPE containing sequences complementary to the 3′-end of the probe, and hydroxyapatite. In a specific embodiment, if a dual labeled oligonucleotide comprising a 3′ biotin label separated from a 5′ label by a nuclease susceptible cleavage site is employed as the signal means, the PCR-amplified mixture may be contacted with materials containing a specific binding partner such as avidin or streptavidin, or an antibody or monoclonal antibody to biotin. Such materials may include beads and particles coated with specific binding partners and may also include magnetic particles.

In some embodiments, after the PCR mixture has been contacted with an SPE, the SPE material may be removed by filtration, sedimentation, or magnetic attraction, leaving the labeled fragments free of uncleaved labeled oligonucleotides and available for detection.

The resultant PCR product may be detected using, for example, agarose gel electrophoresis. Alternatively, the resultant products of the amplification reaction may be detected using a detectable label, that is, e.g., isotopic, fluorescent, colorimetric, and/or otherwise detectable, e.g., using antibodies. According to some embodiments, amplification methods of the disclosure may be used to amplify virtually any target nucleic acid such as a nucleic acid fragment, gene fragment (e.g., an exon or intron fragment), cDNA, or chromosomal fragment.

Genotyping by SNP (single nucleotide polymorphism) analysis and allele-specific oligonucleotide (ASO) hybridizations, which may be the basis for microarray or DNA-Chip methods, are other genomic methods that may benefit from a technology for enhanced accuracy of hybridization. Microarrays may be constructed by arraying and linking PCR amplified cDNA clones or genes to a derivatized glass plate. Currently, the linking chemistries may depend on high-salt buffers with formamide or dimethyl sulfoxide (DMSO) to denature the DNA and provide more single-stranded targets for eventual hybridization with high specificity and minimal background. This may be a critical step in the preparation of reproducible, high-fidelity microarrays which may benefit from reversibly modified nucleic acids developed according to some embodiments of the disclosure. Further, the specific conditions of pre-hybridization and hybridization steps may dramatically affect the signal from the microarray. In some embodiments, compositions, systems, and/or methods of the disclosure may improve microarray performance at this step of the process.

Diagnostic Applications

Methods, compositions, systems and kits of the disclosure may be useful in a variety of diagnostic applications, such as, for example, the amplification and/or detection of nucleic acid sequences found in genomic DNA, bacterial DNA, fungal DNA, and/or viral RNA and/or DNA. Compositions, systems and methods, according to some embodiments, may be used to detect and/or characterize nucleic acid sequences associated with infectious diseases (e.g., gonorrhea, chlamydia), genetic disorders, and/or cellular disorders such as cancer; or for the detection of certain types of non-genetic diseases (e.g., to detect the presence of a viral nucleic acid molecule (e.g., HIV or hepatitis) within a nucleic acid sample derived from a human cell sample). Surface analysis, e.g., through the use of microarrays or gene chips, to detect the possible presence of, e.g., biowarfare agents, may be aided through the practice of at least some embodiments of the present disclosure.

Forensic Applications

Forensic science related to the application of experimental techniques of molecular biology, biochemistry, and genetics to the examination of biological evidence for the purpose, for example, of positively identifying the perpetrator of a crime. The sample size of such biological evidence (e.g. hair, skin, blood, saliva, or semen) may be very small and may contain contaminants and/or interferents of molecular assays. Accordingly, compositions, systems, and/or methods may be used to detect, for example, the sex or species of origin of even minute biological samples in some embodiments of the disclosure.

Research Applications

In some embodiments, methods, compositions, and systems of the disclosure may have a variety of research applications. For example, they may be useful for any research application in which genetic analyses must be performed on limited amounts of nucleic acid sample.

In general, the practice at least some embodiments of the present disclosure may employ, unless otherwise indicated, conventional techniques of chemistry, molecular biology, recombinant DNA technology, PCR technology, immunology, and any necessary cell culture or animal husbandry techniques, which are within the skill of the art having the benefit of the instant disclosure.

In some embodiments a method of suppressing the interference of a masking agent on a molecular assay of a nucleic acid-containing test sample may comprise contacting the test sample with buffered solution comprising (a) at least one chelator (e.g., a divergent metal chelator), (b) at least one chelator enhancing component, and (c) at least one buffer, wherein the pH of the buffered solution is from about 4.5 to about 8.0 and wherein the amounts of the divalent metal chelator and the chelator enhancing component are selected such that the interference of the masking agent on the molecular assay is suppressed, for example, relative to a test sample not contacted with the buffered solution.

A nucleic acid test sample may be further contacted with at least one enzyme-inactivating component selected from the group consisting of manganese chloride, sodium lauroyl sarcosinate, and/or sodium dodecyl sulfate in the range of up to about 5% (w/v). Also as described above, the buffered solution may further comprise at least one nonionic detergent.

A nucleic acid in a nucleic acid test sample may comprise, according to some embodiments, eukaryotic DNA, eukaryotic RNA, viral DNA, viral RNA, prokaryotic DNA, prokeryotic RNA, genomic DNA, cDNA, mRNA, artificial DNA, and/or artificial RNA.

A method of improving the signal response of a molecular assay of a nucleic acid-containing test sample, in some embodiments, may comprise the steps of:
(1) contacting a sample containing a nucleic acid with an amount of at least one divalent metal chelator and an amount of at least one chelator enhancing component in a buffered solution comprising at least one buffer such that the pH of the buffered solution is from about 4.5 to about 8.0, the amounts of the divalent metal chelator and the chelator enhancing component being selected such that the interference of the masking agent on the molecular assay is suppressed; and
(2) extracting the nucleic acid from the sample; and
(3) conducting a molecular assay on said extracted nucleic acid, wherein the signal response of said molecular assay is improved. A molecular assay may include PCR, LCR, RT-PCR, NASBA, SDA, LCX, hybridization, and/or genetic transformation testing.

Methods for extracting a nucleic acid from a sample may include extraction with phenol or phenol:chloroform. Phenol-chloroform extraction may be followed by extraction with chloroform (e.g. buffered phenol containing 0.1% hydroxyquinoline in some embodiments). Extraction may also be performed with phenol:chloroform:isoamyl alcohol (25:24:1). Extracted nucleic acids may be precipitated with cold ethanol. Other extraction and purification methods are known in the art.

A method of improving hybridization of a nucleic acid may, in some embodiments, comprise:
(1) contacting a sample containing a nucleic acid with an amount of at least one divalent metal chelator and an amount of at least one chelator enhancing component in a buffered solution comprising at least one buffer such that the pH of the buffered solution is from about 4.5 to about 8.0, the amounts of the divalent metal chelator and the chelator enhancing component being selected such that the interference of the masking agent on hybridization of the nucleic acid is suppressed, such that a test solution for hybridization is formed; and
(2) contacting the test solution with a target nucleic acid under conditions favorable for hybridization, such that hybridization occurs, the interfering effect of a masking agent on the hybridization being reduced or suppressed.

In some embodiments, hybridization may be performed on microarrays and/or DNA chips (e.g., microarrays and/or DNA chips known in the art). The use of microarrays is described in M. Schema, ed., “Microarray Biochip Technology” (Eaton Publishing, 2000), incorporated herein by this reference. Methods for the computer-driven analysis and interpretation of microarray data and its use in bioinformatics are well known in the art.

A test sample, according to some embodiments may comprise a nucleic acid in a buffered solution, the buffered solution comprising at least one buffer such that the pH of the buffered solution is from about 4.5 to about 8.0. A the buffered solution may further comprise an amount of at least one divalent metal chelator and/or an amount of at least one chelator enhancing component, the amounts of the divalent metal chelator and the chelator enhancing component being selected such that the interference of at least one masking agent on a molecular assay performed on the nucleic acid in the test sample is suppressed.

### SOME SPECIFIC EXAMPLE EMBODIMENTS OF THE DISCLOSURE

Some of the various embodiments of compositions, systems, and methods of the disclosure may be described as follows:

1. A method of suppressing the interference of a masking agent on a molecular assay of a nucleic acid-containing test sample comprising the step of contacting the test sample with an amount of at least one divalent metal chelator and an amount of at least one chelator enhancing component in a buffered solution comprising at least one buffer such that the pH of the buffered solution is from about 4.5 to about 8.0, the amounts of the divalent metal chelator and the chelator enhancing component being selected such that the interference of the masking agent on the molecular assay is suppressed.

2. A method according to embodiment 1 wherein the at least one divalent metal chelator is selected from the group consisting of ethylenediaminetetraacetic acid (EDTA); imidazole; [ethylenebis(oxyethylenenitrilo)]tetraacetic acid (EGTA); iminodiacetate (IDA); 1,2-bis(2-aminophenoxy)ethane-N,N,N′,N′-tetraacetic acid (BAPTA); bis(5-amidino-2-benzimidazolyl)methane (BABIM) and salts thereof.

3. A method according to embodiment 2 wherein the at least one divalent metal chelator is selected from the group consisting of EDTA, EGTA and BAPTA.

4. A method according to embodiment 1 wherein the concentration of the at least one divalent metal chelator is from about 0.001 M to about 0.6 M in the test sample.

5. A method according to embodiment 4 wherein the concentration of the at least one divalent metal chelator is from about 0.1 M to about 0.5 M in the test sample.

6. A method according to embodiment 5 wherein the concentration of the at least one divalent metal chelator is from about 0.2 M to about 0.4 M in the test sample.

7. A method according to embodiment 1 wherein the at least one chelator enhancing component is selected from the group consisting of lithium chloride, guanidinium chloride, guanidinium thiocyanate, guanidinium isothiocyanate, sodium salicylate, sodium perchlorate, sodium thiocyanate, and sodium isothiocyanate.

8. A method according to embodiment 1 wherein the concentration of the at least one chelator enhancing component is from about 0.01 M to about 0.9 M in the test sample.

9. A method according to embodiment 8 wherein the concentration of the at least one chelator enhancing component is from about 0.1 M to about 0.8 M in the test sample.

10. A method according to embodiment 9 wherein the concentration of the at least one chelator enhancing component is from about 0.2 M to about 0.7 M in the test sample.

11. A method according to embodiment 1 wherein the buffer is selected from the group consisting of potassium acetate, sodium acetate, potassium phosphate, sodium phosphate, tris(hydroxymethyl)aminomethane (Tris), (N-(2-hydroxyethyl)piperazine-N′-(2-ethanesulfonic acid) (HEPES), MOPS buffer (3-(N-morpholino)propanesulfonic acid), ACES (2-[(2-amino-2-oxoethyl)amino]ethanoesulfonic acid) buffer, ADA (N-(2-acetamido)2-iminodiacetic acid) buffer, AMPSO (3-[(1,1-dimethyl-2-hydroxyethyl)amino]-2-propanesulfonic acid) buffer, BES (N,N-bis(2-hydroxyethyl)-2 aminoethanesulfonic acid buffer, Bicine (N,N-bis(2-hydroxyethylglycine) buffer, Bis-Tris (bis-(2-hydroxyethyl)imino-tris(hydroxymethyl)methane buffer, CAPS (3-(cyclohexylamino)-1-propanesulfonic acid) buffer, CAPSO (3-(cyclohexylamino)-2-hydroxy-1-propanesulfonic acid) buffer, CHES (2-(N-cyclohexylamino)ethanesulfonic acid) buffer, DIPSO (3-[N,N-bis(2-hydroxyethyl)amino]-2-hydroxy-propanesulfonic acid) buffer, HEPPS(N-(2-hydroxyethylpiperazine)-N′-(3-propanesulfonic acid) buffer, HEPPSO(N-(2-hydroxyethyl)piperazine-N′-(2-hydroxypropanesulfonic acid) buffer, MES (2-(N-morpholino)ethanesulfonic acid) buffer, triethanolamine buffer, imidazole buffer, glycine buffer, ethanolamine buffer, phosphate buffer, MOPSO (3-(N-morpholino)-2-hydroxypropanesulfonic acid) buffer, PIPES (piperazine-N,N′-bis(2-ethanesulfonic acid) buffer, POPSO (piperazine-N,N′-bis(2-hydroxypropaneulfonic acid) buffer; TAPS(N-tris[hydroxymethyl)methyl-3-aminopropanesulfonic acid) buffer, TAPSO (3-[N-tris(hydroxymethyl)methylamino]-2-hydroxy-propanesulfonic acid) buffer, TES (N-tris(hydroxymethyl)methyl-2-aminoethanesulfonic acid) buffer, tricine (N-tris(hydroxymethyl)methylglycine buffer), 2-amino-2-methyl-1,3-propanediol buffer, and 2-amino-2-methyl-1-propanol buffer.

12. A method according to embodiment 11 wherein the buffer is selected from the group consisting of potassium acetate, sodium acetate, potassium phosphate, sodium phosphate, Tris, and HEPES.

13. A method according to embodiment 1 wherein the pH of the buffered solution is from about 4.5 to about 7.8, from about 4.5 to about 6.9, and/or from about 6.9 to about 7.6.

14. A method according to embodiment 1 wherein the masking agent is selected from the group consisting of leukocyte esterases, heme proteins, and myoglobin and hemoglobin analogs, derivatives, oxidation and breakdown products.

15. A method according to embodiment 14 wherein the masking agent is selected from the group consisting of ferritins, methemoglobin, sulfhemoglobin and bilirubin.

16. A method according to embodiment 15 wherein the masking agent is selected from the group consisting of methemoglobin and bilirubin.

17. A method according to embodiment 1 wherein the nucleic-acid containing test sample is further contacted with at least one enzyme-inactivating component selected from the group consisting of manganese chloride, sodium lauroyl sarcosinate, and sodium dodecyl sulfate in the range of up to about 5% (w/v) concentration in the test sample.

18. A method according to embodiment 1 wherein the buffered solution further comprises at least one nonionic detergent.

19. A method according to embodiment 18 wherein the at least one nonionic detergent is selected from the group consisting of polyoxyethylene sorbitan monolaurates, octyl- and nonyl-phenoxypolyethoxylethanols (Nonidet detergents), octyl glucopyranosides, dodecyl maltopyranosides, heptyl thioglucopyranosides, Big CHAP detergents, Genapol X-80, Pluronic detergents, polyoxyethylene esters of alkylphenols (Triton), and derivatives and analogues thereof.

20. A method according to embodiment 19 wherein the at least one nonionic detergent is a polyoxyethylene sorbitan monolaurate.

21. A method according to embodiment 20 wherein the polyoxyethylene sorbitan monolaurate is polyoxyethylene (20) sorbitan monolaurate.

22. A method according to embodiment 21 wherein the concentration of polyoxyethylene (20) sorbitan monolaurate is about 0.01% (w/v) in the test sample.

23. A method according to embodiment 1 wherein the nucleic acid is DNA.

24. A method according to embodiment 23 wherein the DNA is eukaryotic DNA.

25. A method according to embodiment 23 wherein the DNA is cDNA.

26. A method according to embodiment 1 wherein the nucleic acid is RNA.

27. A method according to embodiment 26 wherein the RNA is mRNA.

28. A method of improving the signal response of a molecular assay of a nucleic acid-containing test sample comprising the steps of:

(a) contacting a sample containing a nucleic acid with an amount of at least one divalent metal chelator and an amount of at least one chelator enhancing component in a buffered solution comprising at least one buffer such that the pH of the buffered solution is from about 4.5 to about 8.0, the amounts of the divalent metal chelator and the chelator enhancing component being selected such that the interference of the masking agent on the molecular assay is suppressed; and

(b) extracting the nucleic acid from the sample; and

(c) conducting a molecular assay on said extracted nucleic acid, wherein the signal response of said molecular assay is improved.

29. A method according to embodiment 28 wherein the molecular assay is selected from the group consisting of the polymerase chain reaction, the ligase amplification reaction, RT-PCR, NASBA, SDA, LCₓ, hybridization, and genetic transformation testing.

30. A method according to embodiment 29 wherein the molecular assay is the polymerase chain reaction.

31. A method according to embodiment 28 wherein the sample containing the nucleic acid is a bodily fluid.

32. A method according to embodiment 31 wherein the bodily fluid is selected from the group consisting of urine, blood, blood serum, amniotic fluid; cerebrospinal fluid, spinal fluid, synovial fluid, conjunctival fluid, salivary fluid, vaginal fluid, stool, seminal fluid, lymph, bile, tears, and sweat.

33. A method according to embodiment 32 wherein the bodily fluid is urine.

34. A method according to embodiment 28 wherein the at least one divalent metal chelator is selected from the group consisting of ethylenediaminetetraacetic acid (EDTA); imidazole; [ethylenebis(oxyethylenenitrilo)]tetraacetic acid (EGTA); iminodiacetate (IDA); 1,2-bis(2-aminophenoxy)ethane-N,N,N′,N′-tetraacetic acid (BAPTA); bis(5-amidino-2-benzimidazolyl)methane (BABIM) and salts thereof.

35. A method according to embodiment 34 wherein the at least one divalent metal chelator is selected from the group consisting of EDTA, EGTA and BAPTA.

36. A method according to embodiment 28 wherein the concentration of the at least one divalent metal chelator is from about 0.001 M to about 0.6 M in the test sample.

37. A method according to embodiment 36 wherein the concentration of the at least one divalent metal chelator is from about 0.1 M to about 0.5 M in the test sample.

38. A method according to embodiment 37 wherein the concentration of the at least one divalent metal chelator is from about 0.2 M to about 0.4 M in the test sample.

39. A method according to embodiment 28 wherein the at least one chelator enhancing component is selected from the group consisting of lithium chloride, guanidinium chloride, guanidinium thiocyanate, guanidinium isothiocyanate, sodium salicylate, sodium perchlorate, sodium thiocyanate, and sodium isothiocyanate.

40. A method according to embodiment 28 wherein the concentration of the at least one chelator enhancing component is from about 0.01 M to about 0.9 M in the test sample.

41. A method according to embodiment 40 wherein the concentration of the at least one chelator enhancing component is from about 0.1 M to about 0.8 M in the test sample.

42. A method according to embodiment 41 wherein the concentration of the at least one chelator enhancing component is from about 0.2 M to about 0.7 M in the test sample.

43. A method according to embodiment 28 wherein the buffer is selected from the group consisting of potassium acetate, sodium acetate, potassium phosphate, sodium phosphate, tris(hydroxymethyl)aminomethane (Tris), (N-(2-hydroxyethyl)piperazine-N′-(2-ethanesulfonic acid) (HEPES), MOPS buffer (3-(N morpholino)propanesulfonic acid), ACES (2-[(2-amino-2-oxoethyl)amino]ethanoesulfonic acid) buffer, ADA (N-(2-acetamido)2-iminodiacetic acid) buffer, AMPSO (3-[(1,1-dimethyl-2-hydroxyethyl)amino]-2-propanesulfonic acid) buffer, BES (N,N-bis(2-hydroxyethyl)-2-aminoethanesulfonic acid buffer, Bicine (N,N-bis(2-hydroxyethylglycine) buffer, Bis-Tris (bis-(2-hydroxyethyl)imino-tris(hydroxymethyl)methane buffer, CAPS (3-(cyclohexylamino)-1-propanesulfonic acid) buffer, CAPSO (3-(cyclohexylamino)-2-hydroxy-1-propanesulfonic acid) buffer, CHES (2-(N-cyclohexylamino)ethanesulfonic acid) buffer, DIPSO (3-[N,N-bis(2-hydroxyethyl)amino]-2-hydroxy-propanesulfonic acid) buffer, HEPPS(N-(2-hydroxyethylpiperazine)-N′-(3-propanesulfonic acid) buffer, HEPPSO(N-(2-hydroxyethyl)piperazine-N′-(2-hydroxypropanesulfonic acid) buffer, MES (2-(N-morpholino)ethanesulfonic acid) buffer, triethanolamine buffer, imidazole buffer, glycine buffer, ethanolamine buffer, phosphate buffer, MOPSO (3-(N-morpholino)-2-hydroxypropanesulfonic acid) buffer, PIPES (piperazine-N,N′-bis(2-ethanesulfonic acid) buffer, POPSO (piperazine-N,N′-bis(2-hydroxypropaneulfonic acid) buffer; TAPS(N-tris[hydroxymethyl)methyl-3-aminopropanesulfonic acid) buffer, TAPSO (3-[N-tris(hydroxymethyl)methylamino]-2-hydroxy-propanesulfonic acid) buffer, TES (N-tris(hydroxymethyl)methyl-2-aminoethanesulfonic acid) buffer, tricine (N-tris(hydroxymethyl)methylglycine buffer), 2-amino-2-methyl-1,3-propanediol buffer, and 2-amino-2-methyl-1-propanol buffer.

44. A method according to embodiment 43 wherein the buffer is selected from the group consisting of potassium acetate, sodium acetate, potassium phosphate, sodium phosphate, Tris, and HEPES.

45. A method according to embodiment 28 wherein the pH of the buffered solution is from about 4.5 to about 7.8, from about 4.5 to about 6.9, and/or from about 6.9 to about 7.6.

46. A method according to embodiment 28 wherein the masking agent is selected from the group consisting of leukocyte esterases, heme proteins, and myoglobin and hemoglobin analogs, derivatives, oxidation and breakdown products.

47. A method according to embodiment 46 wherein the masking agent is selected from the group consisting of ferritins, methemoglobin, sulfhemoglobin and bilirubin.

48. A method according to embodiment 47 wherein the masking agent is selected from the group consisting of methemoglobin and bilirubin.

49. A method according to embodiment 28 wherein the nucleic-acid containing sample is further contacted with at least one enzyme-inactivating component selected from the group consisting of manganese chloride, sodium lauroyl sarcosinate, and sodium dodecyl sulfate in the range of up to about 5% (w/v) concentration in the test sample.

50. A method according to embodiment 28 wherein the buffered solution further comprises at least one nonionic detergent.

51. A method according to embodiment 50 wherein the at least one nonionic detergent is selected from the group consisting of polyoxyethylene sorbitan monolaurates, octyl- and nonyl-phenoxypolyethoxylethanols (Nonidet detergents), octyl glucopyranosides, dodecyl maltopyranosides, heptyl thioglucopyranosides, Big CHAP detergents, Genapol X-80, Pluronic detergents, polyoxyethylene esters of alkylphenols (Triton), and derivatives and analogues thereof.

52. A method according to embodiment 51 wherein the at least one nonionic detergent is a polyoxyethylene sorbitan monolaurate.

53. A method according to embodiment 52 wherein the polyoxyethylene sorbitan monolaurate is polyoxyethylene (20) sorbitan monolaurate.

54. A method according to embodiment 53 wherein the concentration of polyoxyethylene (20) sorbitan monolaurate is about 0.01% (w/v) in the test sample.

55. A method according to embodiment 28 wherein the nucleic acid is DNA.

56. A method according to embodiment 55 wherein the DNA is eukaryotic DNA.

57. A method according to embodiment 55 wherein the DNA is cDNA.

58. A method according to embodiment 28 wherein the nucleic acid is RNA.

59. A method according to embodiment 58 wherein the RNA is mRNA.

60. A method of improving hybridization of a nucleic acid comprising the steps of:

(a) contacting a sample containing a nucleic acid with an amount of at least one divalent metal chelator and an amount of at least one chelator enhancing component in a buffered solution comprising at least one buffer such that the pH of the buffered solution is from about 4.5 to about 8.0, the amounts of the divalent metal chelator and the chelator enhancing component being selected such that the interference of the masking agent on hybridization of the nucleic acid is suppressed, such that a test solution for hybridization is formed; and

(b) contacting the test solution with a target nucleic acid under conditions favorable for hybridization, such that hybridization occurs, the interfering effect of a masking agent on the hybridization being reduced or suppressed.

61. A method according to embodiment 60 wherein the nucleic acid is DNA.

62. A method according to embodiment 61 wherein the DNA is eukaryotic DNA.

63. A method according to embodiment 61 wherein the DNA is cDNA.

64. A method according to embodiment 60 wherein the nucleic acid is RNA.

65. A method according to embodiment 64 wherein the RNA is mRNA.

66. A method according to embodiment 60 wherein the at least one divalent metal chelator is selected from the group consisting of ethylenediaminetetraacetic acid (EDTA); imidazole; [ethylenebis(oxyethylenenitrilo)]tetraacetic acid (EGTA); iminodiacetate (IDA); 1,2-bis(2-aminophenoxy)ethane-N,N,N′,N′-tetraacetic acid (BAPTA); bis(5-amidino-2-benzimidazolyl)methane (BABIM) and salts thereof.

67. A method according to embodiment 66 wherein the at least one divalent metal chelator is selected from the group consisting of EDTA, EGTA and BAPTA.

68. A method according to embodiment 66 wherein the concentration of the at least one divalent metal chelator is from about 0.001 M to about 0.6 M in the test sample.

69. A method according to embodiment 68 wherein the concentration of the at least one divalent metal chelator is from about 0.1 M to about 0.5 M in the test sample.

70. A method according to embodiment 69 wherein the concentration of the at least one divalent metal chelator is from about 0.2 M to about 0.4 M in the test sample.

71. A method according to embodiment 60 wherein the at least one chelator enhancing component is selected from the group consisting of lithium chloride, guanidinium chloride, guanidinium thiocyanate, guanidinium isothiocyanate, sodium salicylate, sodium perchlorate, sodium thiocyanate, and sodium isothiocyanate.

72. A method according to embodiment 60 wherein the concentration of the at least one chelator enhancing component is from about 0.01 M to about 0.9 M in the test sample.

73. A method according to embodiment 72 wherein the concentration of the at least one chelator enhancing component is from about 0.1 M to about 0.8 M in the test sample.

74. A method according to embodiment 73 wherein the concentration of the at least one chelator enhancing component is from about 0.2 M to about 0.7 M in the test sample.

75. A method according to embodiment 60 wherein the buffer is selected from the group consisting of potassium acetate, sodium acetate, potassium phosphate, sodium phosphate, tris(hydroxymethyl)aminomethane (Tris), (N-(2-hydroxyethyl)piperazine-N′-(2-ethanesulfonic acid) (HEPES), MOPS buffer (3-(N morpholino)propanesulfonic acid), ACES (2-[(2-amino-2-oxoethyl)amino]ethanoesulfonic acid) buffer, ADA (N-(2-acetamido)2-iminodiacetic acid) buffer, AMPSO (3-[(1,1-dimethyl-2-hydroxyethyl)amino]-2-propanesulfonic acid) buffer, BES (N,N-bis(2-hydroxyethyl)-2-aminoethanesulfonic acid buffer, Bicine (N,N-bis(2-hydroxyethylglycine) buffer, Bis-Tris (bis-(2-hydroxyethyl)imino-tris(hydroxymethyl)methane buffer, CAPS (3-(cyclohexylamino)-1-propanesulfonic acid) buffer, CAPSO (3-(cyclohexylamino)-2-hydroxy-1-propanesulfonic acid) buffer, CHES (2-(N-cyclohexylamino)ethanesulfonic acid) buffer, DIPSO (3-[N,N-bis(2-hydroxyethyl)amino]-2-hydroxy-propanesulfonic acid) buffer, HEPPS(N-(2-hydroxyethylpiperazine)-N′-(3-propanesulfonic acid) buffer, HEPPSO(N-(2-hydroxyethyl)piperazine-N′-(2-hydroxypropanesulfonic acid) buffer, MES (2-(N-morpholino)ethanesulfonic acid) buffer, triethanolamine buffer, imidazole buffer, glycine buffer, ethanolamine buffer, phosphate buffer, MOPSO (3-(N-morpholino)-2-hydroxypropanesulfonic acid) buffer, PIPES (piperazine-N,N′-bis(2-ethanesulfonic acid) buffer, POPSO (piperazine-N,N′-bis(2-hydroxypropaneulfonic acid) buffer; TAPS(N-tris[hydroxymethyl)methyl-3-aminopropanesulfonic acid) buffer, TAPSO (3-[N-tris(hydroxymethyl)methylamino]-2-hydroxy-propanesulfonic acid) buffer, TES (N-tris(hydroxymethyl)methyl-2-aminoethanesulfonic acid) buffer, tricine (N-tris(hydroxymethyl)methylglycine buffer), 2-amino-2-methyl-1,3-propanediol buffer, and 2-amino-2-methyl-1-propanol buffer.

76. A method according to embodiment 75 wherein the buffer is selected from the group consisting of potassium acetate, sodium acetate, potassium phosphate, sodium phosphate, Tris, and HEPES.

77. A method according to embodiment 60 wherein the pH of the buffered solution is from about 4.5 to about 7.8, from about 4.5 to about 6.9, and/or from about 6.9 to about 7.6.

78. A method according to embodiment 60 wherein the masking agent is selected from the group consisting of leukocyte esterases, heme proteins, and myoglobin and hemoglobin analogs, derivatives, oxidation and breakdown products.

79. A method according to embodiment 78 wherein the masking agent is selected from the group consisting of ferritins, methemoglobin, sulfhemoglobin and bilirubin.

80. A method according to embodiment 79 wherein the masking agent is selected from the group consisting of methemoglobin and bilirubin.

81. A method according to embodiment 60 wherein the nucleic-acid containing sample is further contacted with at least one enzyme-inactivating component selected from the group consisting of manganese chloride, sodium lauroyl sarcosinate, and sodium dodecyl sulfate in the range of up to about 5% (w/v) concentration in the test sample.

82. A method according to embodiment 60 wherein the buffered solution further comprises at least one nonionic detergent.

83. A method according to embodiment 82 wherein the at least one nonionic detergent is selected from the group consisting of polyoxyethylene sorbitan monolaurates, octyl- and nonyl-phenoxypolyethoxylethanols (Nonidet detergents), octyl glucopyranosides, dodecyl maltopyranosides, heptyl thioglucopyranosides, Big CHAP detergents, Genapol X-80, Pluronic detergents, polyoxyethylene esters of alkylphenols (Triton), and derivatives and analogues thereof.

84. A method according to embodiment 83 wherein the at least one nonionic detergent is a polyoxyethylene sorbitan monolaurate.

85. A method according to embodiment 84 wherein the polyoxyethylene sorbitan monolaurate is polyoxyethylene (20) sorbitan monolaurate.

86. A method according to embodiment 85 wherein the concentration of polyoxyethylene (20) sorbitan monolaurate is about 0.01% (w/v) in the test sample.

87. A test sample comprising nucleic acid in a buffered solution, the buffered solution comprising at least one buffer such that the pH of the buffered solution is from about 4.5 to about 8.0, the buffered solution further comprising an amount of at least one divalent metal chelator and an amount of at least one chelator enhancing component, the amounts of the divalent metal chelator and the chelator enhancing component being selected such that the interference of at least one masking agent on a molecular assay performed on the nucleic acid in the test sample is suppressed.

88. A test sample according to embodiment 87 wherein the nucleic acid is DNA.

89. A test sample according to embodiment 88 wherein the DNA is eukaryotic DNA.

90. A test sample according to embodiment 88 wherein the DNA is cDNA.

91. A test sample according to embodiment 87 wherein the nucleic acid is RNA.

92. A test sample according to embodiment 91 wherein the RNA is mRNA.

93. A test sample according to embodiment 87 wherein the at least one divalent metal chelator is selected from the group consisting of ethylenediaminetetraacetic acid (EDTA); imidazole; [ethylenebis(oxyethylenenitrilo)]tetraacetic acid (EGTA); iminodiacetate (IDA); 1,2-bis(2-aminophenoxy)ethane-N,N,N′,N′-tetraacetic acid (BAPTA); bis(5-amidino-2-benzimidazolyl)methane (BABIM) and salts thereof.

94. A test sample according to embodiment 93 wherein the at least one divalent metal chelator is selected from the group consisting of EDTA, EGTA and BAPTA.

95. A test sample according to embodiment 93 wherein the concentration of the at least one divalent metal chelator is from about 0.001 M to about 0.6 M in the test sample.

96. A test sample according to embodiment 95 wherein the concentration of the at least one divalent metal chelator is from about 0.1 M to about 0.5 M in the test sample.

97. A test sample according to embodiment 96 wherein the concentration of the at least one divalent metal chelator is from about 0.2 M to about 0.4 M in the test sample.

98. A test sample according to embodiment 87 wherein the at least one chelator enhancing component is selected from the group consisting of lithium chloride, guanidinium chloride, guanidinium thiocyanate, guanidinium isothiocyanate, sodium salicylate, sodium perchlorate, sodium thiocyanate, and sodium isothiocyanate.

99. A test sample according to embodiment 87 wherein the concentration of the at least one chelator enhancing component is from about 0.01 M to about 0.9 M in the test sample.

100. A test sample according to embodiment 99 wherein the concentration of the at least one chelator enhancing component is from about 0.1 M to about 0.8 M in the test sample.

101. A test sample according to embodiment 100 wherein the concentration of the at least one chelator enhancing component is from about 0.2 M to about 0.7 M in the test sample.

102. A test sample according to embodiment 87 wherein the buffer is selected from the group consisting of potassium acetate, sodium acetate, potassium phosphate, sodium phosphate, tris(hydroxymethyl)aminomethane (Tris), (N-(2-hydroxyethyl)piperazine-N′-(2-ethanesulfonic acid) (HEPES), MOPS buffer (3-(N-morpholino)propanesulfonic acid), ACES (2-[(2-amino-2-oxoethyl)amino]ethanoesulfonic acid) buffer, ADA (N-(2-acetamido)2-iminodiacetic acid) buffer, AMPSO (3-[(1,1-dimethyl-2-hydroxyethyl)amino]-2-propanesulfonic acid) buffer, BES (N,N-bis(2-hydroxyethyl)-2-aminoethanesulfonic acid buffer, Bicine (N,N-bis(2-hydroxyethylglycine) buffer, Bis-Tris (bis-(2-hydroxyethyl)imino-tris(hydroxymethyl)methane buffer, CAPS (3-(cyclohexylamino)-1-propanesulfonic acid) buffer, CAPSO (3-(cyclohexylamino)-2-hydroxy-1-propanesulfonic acid) buffer, CHES (2-(N-cyclohexylamino)ethanesulfonic acid) buffer, DIPSO (3-[N,N-bis(2-hydroxyethyl)amino]-2-hydroxy-propanesulfonic acid) buffer, HEPPS(N-(2-hydroxyethylpiperazine)-N′-(3-propanesulfonic acid) buffer, HEPPSO(N-(2-hydroxyethyl)piperazine-N′-(2-hydroxypropanesulfonic acid) buffer, MES (2-(N-morpholino)ethanesulfonic acid) buffer, triethanolamine buffer, imidazole buffer, glycine buffer, ethanolamine buffer, phosphate buffer, MOPSO (3-(N-morpholino)-2-hydroxypropanesulfonic acid) buffer, PIPES (piperazine-N,N′-bis(2-ethanesulfonic acid) buffer, POPSO (piperazine-N,N′-bis(2-hydroxypropaneulfonic acid) buffer; TAPS(N-tris[hydroxymethyl)methyl-3-aminopropanesulfonic acid) buffer, TAPSO (3-[N-tris(hydroxymethyl)methylamino]-2-hydroxy-propanesulfonic acid) buffer, TES (N-tris(hydroxymethyl)methyl-2-aminoethanesulfonic acid) buffer, tricine (N-tris(hydroxymethyl)methylglycine buffer), 2-amino-2-methyl-1,3-propanediol buffer, and 2-amino-2-methyl-1-propanol buffer.

103. A test sample according to embodiment 102 wherein the buffer is selected from the group consisting of potassium acetate, sodium acetate, potassium phosphate, sodium phosphate, Tris, and HEPES.

104. A test sample according to embodiment 87 wherein the pH of the buffered solution is from about 4.5 to about 7.8, from about 4.5 to about 6.9, and/or from about 6.9 to about 7.6.

105. A test sample according to embodiment 87 wherein the masking agent is selected from the group consisting of leukocyte esterases, heme proteins, and myoglobin and hemoglobin analogs, derivatives, oxidation and breakdown products.

106. A test sample according to embodiment 105 wherein the masking agent is selected from the group consisting of ferritins, methemoglobin, sulfhemoglobin and bilirubin.

107. A test sample according to embodiment 106 wherein the masking agent is selected from the group consisting of methemoglobin and bilirubin.

108. A test sample according to embodiment 87 wherein the test sample further comprises at least one enzyme-inactivating component selected from the group consisting of manganese chloride, sodium lauroyl sarcosinate, and sodium dodecyl sulfate in the range of up to about 5% (w/v) concentration in the test sample.

109. A test sample according to embodiment 87 wherein the buffered solution further comprises at least one nonionic detergent.

110. A test sample according to embodiment 109 wherein the at least one nonionic detergent is selected from the group consisting of polyoxyethylene sorbitan monolaurates, octyl- and nonyl-phenoxypolyethoxylethanols (Nonidet detergents), octyl glucopyranosides, dodecyl maltopyranosides, heptyl thioglucopyranosides, Big CHAP detergents, Genapol X-80, Pluronic detergents, polyoxyethylene esters of alkylphenols (Triton), and derivatives and analogues thereof.

111. A test sample according to embodiment 110 wherein the at least one nonionic detergent is a polyoxyethylene sorbitan monolaurate.

112. A test sample according to embodiment 111 wherein the polyoxyethylene sorbitan monolaurate is polyoxyethylene (20) sorbitan monolaurate.

113. A test sample according to embodiment 112 wherein the concentration of polyoxyethylene (20) sorbitan monolaurate is about 0.01% (w/v) in the test sample.

The present disclosure provides, in some embodiments, compositions, systems, and methods for storing and preserving nucleic acids and suppressing the effect of masking agents so that the nucleic acids can be used in molecular assays such as PCR, the ligand amplification reaction, reverse transcriptase-PCR, or hybridization assays. Thus, improved sensitivity and precision may be achieved in these assays and allows their efficient use for diagnostic, forensic, and/or research purposes. The use of a buffered solution increases the concentration of chelators and chelator enhancing components that may be used without damage to the integrity of the nucleic acid, providing enhanced suppression of interference from masking agents.

Compositions, systems, and methods according to some embodiments of the present disclosure may be used to store and preserve nucleic acids in bodily fluids or other fluids that contain or are believed to contain nucleic acids. They may be used, in some embodiments, together with detergents or other preservatives. According to some embodiments, they may be simple to use. They may be used in the field, where rapid preservation of samples for forensic purposes is critical in some embodiments.

Compositions, systems, and methods according to some embodiments of the present disclosure may possess industrial applicability for preserving and/or storing nucleic acids so that the nucleic acids may be amplified or analyzed.

With respect to ranges of values, the disclosure contemplates each intervening value between the upper and lower limits of the range to at least a tenth of the lower limit's unit, unless the context clearly indicates otherwise. Moreover, the disclosure contemplates any other stated intervening value(s) and range(s) including either or both of the upper and lower limits of the range, unless specifically excluded from the stated range.

One of ordinary skill in the art will also appreciate that methods and materials similar or equivalent to those described herein may also be used to practice or test embodiments of this disclosure.

All the publications cited are incorporated herein by reference in their entireties, including all published patents, patent applications, literature references, as well as those publications that have been incorporated in those published documents. However, to the extent that any publication incorporated herein by reference refers to information to be published, applicants do not admit that any such information published after the filing date of this application to be prior art.

As used in this specification and in the appended claims, singular forms include the plural forms. For example the terms “a,” “an,” and “the” include plural references unless the content clearly dictates otherwise. Additionally, the term “at least’ preceding a series of elements is to be understood as referring to every element in the series. Embodiments of the disclosure illustratively described herein may be practiced with or without any element or elements, limitation or limitations, not specifically disclosed herein. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the disclosure. Thus, it should be understood that although the present disclosure has been elaborated in terms of some specific example embodiments and/or optional features, modification and variation of the embodiments herein disclosed may be resorted by those skilled in the an, and that such modifications and variations are considered to be within the contemplation of the embodiments disclosed herein.

### EXAMPLES

The invention is illustrated by the following Examples. These Examples are included for illustrative purposes only, and are not intended to limit the invention.

### Example 1

### PCR Detection of Penicillinase-Producing Neisseria gonorrhoeae

A PCR signal-enhancing effect of some specific example embodiments of the disclosure is demonstrated by the following example. Four varieties of TEM-encoding plasmids are found in penicillinase-producing Neisseria gonorrhoeae (PPNG). These are the 6.7 kb (4.4 Mda) Asian type, the 5.1 kb (3.2 Mda) African type, the 4.9 kb (3.05-Mda) Toronto type and the 4.8 kb (2.9-Mda) Rio Type. This PCR assay for PPNG takes advantage of the fact that the TEM-1 gene is located close to the end of the transposon Tn2; by the use of one primer in the TEM-1 gene and the other in a sequence beyond the end of Tn2, and common to all four plasmids, a PCR product only from plasmids and not from TEM-1 encoding plasmids was obtained. (Table 1, below) The conditions associated with this protocol were modified to include the reagent of the invention in the hybridization and the treated probe was mixed with the 761-bp amplification product per standard PCR protocol. The results were read by measuring absorbance at 450 nm (A₄₅₀ₙₘ).

Materials and Reagents:

BBL chocolate II agar plates

Sterile Tris Buffer 10 mM Tris (pH 7.4), 1 mM EDTA

0.5-ml Gene Amp reaction tubes

Sterile disposable Pasteur pipette tips

Aerosol-resistant tips

PCR master mix:

50 mM KCL

2 mM MgCl

50 μM each of

Four deoxyribonucleoside triphosphates: (dATP, dCTP, dGTP, and dTTP);

2.5 U of Taq Polymerase (Perkin Elmer);

5% glycerol;

50 pmol each of primers PPNG-L and PNG-R (per 100 μl reaction)

Denaturation solution

1M Na 5×Denhardt's solution

Prehybridization Solution

5×SSC (1×SSC is 0.015 M NaCl plus 0.015 M sodium citrate);

5×Denhardt's solution;

0.05% SDS;

0.1% sodium pyrophosphate, and

100 μg of sonicated salmon sperm DNA per ml.

Hybridization Solution

Same as prehybridization solution but without Denhardt's solution and including 200 μl of a reagent of the invention.

1 ml of a reagent of the invention (1 M guanidine HCl/0.01 M EDTA, “Reagent 1”)

Avidin-HRP peroxidase complex (Zymed)

Magnetic microparticles (Seradyne)

TABLE 1

| | | |
|---|---|---|
| Function | Name | Nucleotide Sequence 5′ to 3′ |

| | | |
|---|---|---|
| Primer | PPNG-L | AGT TAT CTA CAC GAC GG |
| | | (SEQ ID NO: 1) |
| Primer | PPNG-B | GGC GTA CTA TTC ACT CT |
| | | SEQ ID NO: 2) |
| Probe | PPNG-C | GCG TCA GAC CCC TAT CTA TAA ACT C |
| | | SEQ ID NO: 3) |

Methods:

Sample preparation: 2 colonies were picked from a chocolate agar plate. Colonies were suspended in Dl water just prior to setting up PCR. The master mix was prepared according to the recipe above. 5 μl of the freshly prepared bacterial suspension was added to 95 μl of master mix. The DNA was liberated and denatured in a thermocycler using three cycles of 3 min at 94° C. and 3 min at 55° C. The DNA was amplified in the thermal cycler by using a two step profile: a 25 s denaturation at 95° C. and a 25s annealing at 55° C. for a total of thirty cycles. The time was set between the two temperature plateaus to enable the fastest possible annealing between the two temperatures. 15 pmol of labeled (avidin-HRP complex) detection probe PPNG-C was added to the hybridization solution bound to magnetic micro particles with and without the preservative reagent at 37° C. for 1 hour. The control and treated probes were then added to the amplification product and the reaction was calorimetrically detected at 450 nm. The signal obtained from the hybridization probes treated with a composition according to a specific example embodiment of the disclosure was found to be significantly higher than the untreated probes.

### Example 2

Inhibition of amplification may be a significant problem with STD specimens from cervical and/or urethral sites. Estimates of inhibition range from 2-20% for specimens collected with a swab. This experiment compares a novel swab collection device containing a reagent of the invention to a standard dry swab collection device and demonstrates that reagents according to at least some specific example embodiments of the disclosure may be utilized to reduce (e.g., minimize) the effects of inhibition, thereby reducing the incidence of false negative results.

The swab device used was a sterile polyurethane sponge impregnated with 700 μl of the reagent of Example 1, which is housed in the bottom of an empty sterile tube. The specimen is collected on a separate sterile rayon swab and inserted into the above tube (Starplex). Once the swab has been inserted in the tube, the swab comes into contact with the sponge and absorbs the reagent, which treats the specimen accordingly. The control device used for comparison was a standard dry rayon swab in a sterile tube (Copan Diagnostics #155 C-160 C).

Four known amplification assays were included in this study: LCₓ® (Abbott Diagnostics), Probe-Tec® (BD Diagnostic Systems), TMA™ (Gen Probe), and PCR® (Roche Diagnostics). Four separate laboratories were utilized to conduct the experiment, one for each assay platform.

Specimens were collected at four separate STD clinics using best-practice collection methods. At each collection site, 50 patients provided duplicate specimens for an aggregate of 200 treated samples and 200 untreated samples. All samples were transported to the laboratory at room temperature and processed within 8 hours of collection.

Current assay reagents and direction inserts were used to perform the amplification assay. A second amplified assay was utilized to challenge all positives to confirm that they were really true positives. LCₓ was refereed by PCR, and SDA, TMA, and PCR were all refereed by LCₓ. Additionally, all positive extracts that were untreated (dry) were subjected to GC/MS analysis to confirm the presence of substances known to cause inhibition in amplified assay systems. Target substances were leukocyte esterase, methemoglobin, lactoferrin, hydrogen peroxide, and lactic acid. Furthermore, immunoassays were preformed to detect the presence of the following inhibitors:

Gamma interferon

Mucosal IgA

Non-target bacterial DNA

Data:

1) Comparison Between True Positives Using Reagent 1 and an Untreated Control

Number of collection sites: 4

Collection site 1: Cervical Chlamydia (asymptomatic)

Collection site 2: Urethral Gonorrhea (symptomatic)

Collection site 3: Cervical Chlamydia (asymptomatic)

Collection site 4: Urethral Gonorrhea (symptomatic)

Number of Samples that were Treated: 200 (50 from each collection site).

Number of Samples that were untreated: 200 (50 from each collection site).

TABLE 2

| | | Positives- | | Number | Positives- | |
|---|---|---|---|---|---|---|
| Test Site #/ | Number of | (Treated | | of | Untreated | |
| Assay | Samples | w/Reagent 1) | Prevalence | Samples | control | Prevalence |
| 1 - LCₓ | 50 | 8 | 16% | 50 | 6 | 12% |
| 2 - Probe-Tec | 50 | 7 | 14% | 50 | 4 | 8% |
| 3 - TMA | 50 | 5 | 10% | 50 | 3 | 6% |
| 4 - PCR | 50 | 6 | 12% | 50 | 3 | 6% |
| Totals: | 200 | 26 | 13% | 200 | 16 | 8% |

2) GC/MS Cervical Data for Untreated Inhibited Specimens:

Lactoferrin>175 μg/mg

Methemoglobin>8 mg/dl

Leukocyte esterase>15 μL

Lactic Acid: present, but not quantified

*All had statistically significant correlation with inhibited specimens

3) GC/MS Urethral Data for Untreated Inhibited Specimens:

Neutrophil Esterase>15 μl (achieved peaks)

Hydrogen peroxide: present, but not quantified

Zinc 110 pg/dl

*All had statistically significant correlation with inhibited specimens

4) Immunoassay Data for Untreated Inhibited Specimens:

IgA cervical correlation

Gamma Interferon urethral and cervical correlation

Protein oxidation (hydroxy-nonenal) activity urethral correlation

Results

1) Swabs impregnated with Reagent 1 yielded a statistically significant increase in amplification at all sites compared to a standard untreated swab.

2) There was no statistically significant difference between gonorrhea and chlamydia specimens with regard to their inhibition characteristics.

3) There was a statistically significant presence of target inhibitors in both untreated gonorrhea and chlamydia specimens.

4) Lactoferrin, hydrogen peroxide, methemoglobin, gamma interferon, lactic acid, leukocyte esterase were all associated with inhibited specimens.

### Example 3

### Use of Buffers to Prevent High Molecular Concentrations of Chaotropes from Destroying DNA Sequences of Momp from Chlamydia trachomatis

This example clearly shows that buffered chemistry in at least some specific example embodiments prevents high molar concentrations of chaotropes from destroying the DNA sequences of MOMP (outer membrane protein) from Chlamydia trachomatis and allows these DNA sequences to be amplified effectively by PCR.

Reagent 1 of Example 1 was modified by introducing a higher concentration of chaotrope and chelator and a quantity of one of the following buffers (Buffers I-V) as follows:

Buffer I was HeBS (HEPES-buffered saline solution, pH 7.05, which was prepared by mixing 16.4 g of NaCl, 11.9 g of HEPES acid, 0.21 g of Na₂HPO4, and 800 ml H₂O, and titrating to pH 7.05 with 5 N NaOH.

Buffer II was 0.1 M potassium acetate buffer, prepared by mixing 14.8 ml of Solution A (11.55 ml glacial acetic acid/liter (0.2 M)) and 35.2 ml of Solution B (19.6 g potassium acetate (0.2 M)) to achieve a final pH of 5.0.

Buffer III was 0.1 M sodium phosphate buffer, prepared by mixing 39.0 ml of Solution A (27.6 g NaH₂PO₄.H₂O/liter) and 55.0 ml of Solution B (53.6 g of Na₂HPO₄.7H₂O/liter) to achieve a final pH of 6.9.

Buffer IV was Tris-buffered saline (TBS), containing 100 mM Tris-HCl and 0.9% NaCl, to achieve a final pH of 7.5.

Buffer V was Tween 20/TBS, prepared by adding 0.1% Tween 20 in Tris-buffered saline to TBS Buffer, to achieve a final pH of 7.1.

The following combinations of chelators, chaotropes, and buffers were used:
(1) 2 M EGTA and 3 M guanidinium thiocyanate, not buffered;
(2) 2 M EDTA and 6 M guanidinium chloride, not buffered;
(3) 3 M EGTA and 4 M sodium thiocyanate, not buffered;
(4) 3 M BAPTA and 7 M lithium chloride, not buffered;
(5) 4 M EDTA and 6 M sodium perchlorate, not buffered;
(6) 2 M EGTA and 3 M guanidinium thiocyanate, Buffer I;
(7) 2 M EDTA and 4 M guanidinium chloride, Buffer II;
(8) 3 M EGTA and 6 M sodium thiocyanate, Buffer III;
(9) 3 M BAPTA and 4 M lithium chloride, Buffer IV; and
(10) 4 M EDTA and 7 M sodium perchlorate, Buffer V.

Samples of fresh urine spiked with 100 copies of chlamydia DNA and one of the above combinations of chelators, chaotropes, and buffers were incubated for 1, 2, 3, 4, 5, 6, or 7 hours at 30° C. Subsequent to the incubation, PCR was performed as in Example 1 to detect DNA sequences encoding MOMP (outer membrane protein) of Chlamydia trachomatis.

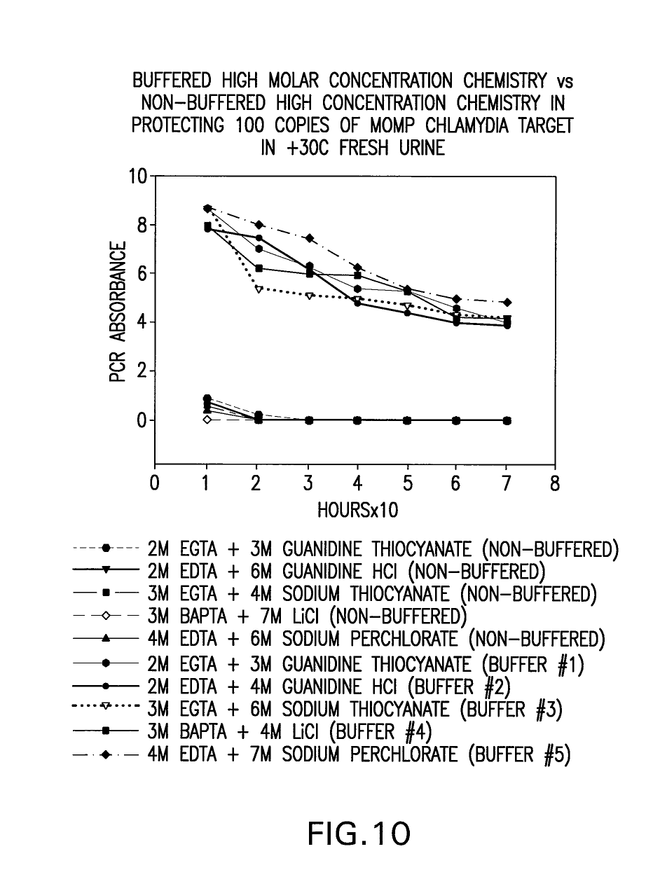

The results are shown in FIG. 10. The results clearly show that the buffered compositions tested prevent high molecular concentrations of chaotropes from destroying specific DNA sequences, allowing the use of these high molecular concentrations of chaotropes to preserve the nucleic acids in the sample and more effectively suppress the effect of masking agents on subsequent assays or procedures such as hybridization or PCR.

What is claimed is:

1. A method of hybridizing a first and second nucleic acid, the method comprising:
(a) contacting
(i) a sample comprising a first nucleic acid and at least one masking agent selected from the group consisting of a leukocyte esterase, a myoglobin analogue, a hemoglobin analogue, a myoglobin derivative, a hemoglobin derivative, a myoglobin oxidation product, a hemoglobin oxidation product, a myoglobin breakdown product, a hemoglobin breakdown product, a ferritin, methemoglobin, sulfhemoglobin, and bilirubin with
(ii) a suppressant composition comprising:
a chelator selected from the group consisting of ethylenediaminetetraacetic acid (EDTA); imidazole; [ethylenebis(oxyethylenenitrilo)]tetraacetic acid (EGTA); iminodiacetate (IDA); 1,2-bis(2-aminophenoxy)ethane-N,N,N′,N′-tetraacetic acid (BAPTA); bis(5-amidino-2-benzimidazolyl)methane (BABIM) and salts thereof;
a chelator enhancing component selected from the group consisting of lithium chloride, sodium salicylate, sodium perchlorate, sodium thiocyanate, and combinations thereof; and
a buffer,
to form a hybridization test solution; and
(b) contacting the hybridization test solution with a second nucleic acid under conditions that permit hybridization of the first and second nucleic acids,
wherein the concentration of the chelator in the hybridization test solution is from about 0.2 M to about 0.6 M,
wherein the concentration of the chelator enhancing component in the hybridization test solution is from about 0.1 M to 0.9 M,
wherein the pH of the hybridization test solution is from about 4.5 to about 7.8, and
wherein the extent of hybridization between the first and second nucleic acids is greater in the presence of the suppressant composition than the extent of hybridization between the first and second nucleic acids in the absence of the suppressant composition.

2. A method according to claim 1, wherein the buffer is selected from the group consisting of potassium acetate, sodium acetate, potassium phosphate, sodium phosphate, tris(hydroxymethyl)aminomethane (Tris), (N-(2-hydroxyethyl)piperazine-N′-(2-ethanesulfonic acid) (HEPES), MOPS buffer (3-(N-morpholino)propanesulfonic acid), ACES (2-[(2-amino-2-oxoethyl)amino]ethanoesulfonic acid) buffer, ADA (N-(2-acetamido)2-iminodiacetic acid) buffer, AMPSO (3-[(1,1-dimethyl-2-hydroxyethyl)amino]-2-propanesulfonic acid) buffer, BES (N,N-bis(2-hydroxyethyl)-2 aminoethanesulfonic acid buffer, Bicine (N,N-bis(2-hydroxyethylglycine) buffer, Bis-Tris (bis-(2-hydroxyethyl)imino-tris(hydroxymethyl)methane buffer, CAPS (3-(cyclohexylamino)-1-propanesulfonic acid) buffer, CAPSO (3-(cyclohexylamino)-2-hydroxy-1-propanesulfonic acid) buffer, CHES (2-(N-cyclohexylamino)ethanesulfonic acid) buffer, DIPSO (3-[N,N-bis(2-hydroxyethyl)amino]-2-hydroxy-propanesulfonic acid) buffer, HEPPS(N-(2-hydroxyethylpiperazine)-N′-(3-propanesulfonic acid) buffer, HEPPSO(N-(2-hydroxyethyl)piperazine-N′-(2-hydroxypropanesulfonic acid) buffer, MES (2-(N-morpholino)ethanesulfonic acid) buffer, triethanolamine buffer, imidazole buffer, glycine buffer, ethanolamine buffer, phosphate buffer, MOPSO (3-(N-morpholino)-2-hydroxypropanesulfonic acid) buffer, PIPES (piperazine-N,N′-bis(2-ethanesulfonic acid) buffer, POPSO (piperazine-N,N′-bis(2-hydroxypropaneulfonic acid) buffer; TAPS(N-tris[hydroxymethyl)methyl-3-aminopropanesulfonic acid) buffer, TAPSO (3-[N-tris(hydroxymethyl)methylamino]-2-hydroxy-propanesulfonic acid) buffer, TES (N-tris(hydroxymethyl)methyl-2-aminoethanesulfonic acid) buffer, tricine (N-tris(hydroxymethyl)methylglycine buffer), 2-amino-2-methyl-1,3-propanediol buffer, 2-amino-2-methyl-1-propanol buffer, and combinations thereof.

3. A method according to claim 1 further comprising contacting the hybridization test solution with at least one enzyme-inactivating component selected from the group consisting of manganese chloride, sodium lauroyl sarcosinate, and sodium dodecyl sulfate in the range of up to about 5% (w/v) concentration in the test sample.

4. A method according to claim 1 wherein the suppressant composition further comprises at least one nonionic detergent is selected from the group consisting of polyoxyethylene sorbitan monolaurates, octyl- and nonyl-phenoxypolyethoxylethanols (Nonidet detergents), octyl glucopyranosides, dodecyl maltopyranosides, heptyl thioglucopyranosides, Big CHAP detergents, Genapol X-80, Pluronic detergents, polyoxyethylene esters of alkylphenols (Triton), and derivatives and analogues thereof.

5. A method of suppressing the interference of a masking agent selected from the group consisting of a leukocyte esterase, a myoglobin analogue, a hemoglobin analogue, a myoglobin derivative, a hemoglobin derivative, a myoglobin oxidation product, a hemoglobin oxidation product, a myoglobin breakdown product, a hemoglobin breakdown product, a ferritin, methemoglobin, sulfhemoglobin, and bilirubin, on a molecular assay of a nucleic acid-containing test sample, the method comprising:
contacting the nucleic acid-containing test sample comprising a masking agent with a suppressant composition comprising:
a chelator selected from the group consisting of ethylenediaminetetraacetic acid (EDTA); imidazole; [ethylenebis(oxyethylenenitrilo)]tetraacetic acid (EGTA); iminodiacetate (IDA); 1,2-bis(2-aminophenoxy)ethane-N,N,N′,N′-tetraacetic acid (BAPTA); bis(5-amidino-2-benzimidazolyl)methane (BABIM) and salts thereof;
a chelator enhancing component selected from the group consisting of lithium chloride, sodium salicylate, sodium perchlorate, sodium thiocyanate, and combinations thereof; and
a buffer,
wherein a nucleic-acid-containing test sample-suppressant composition mixture is formed,
wherein the concentration of the chelator in the mixture is from about 0.2 M to about 0.6 M,
wherein the concentration of the chelator enhancing component in the mixture is from about 0.1 M to 0.9M,
wherein the pH of the mixture is from about 4.5 to about 7.8, and
wherein the interference of the masking agent on the molecular assay of the nucleic acid-containing test sample is suppressed.

6. A method according to claim 5, wherein the buffer is selected from the group consisting of potassium acetate, sodium acetate, potassium phosphate, sodium phosphate, tris(hydroxymethyl)aminomethane (Tris), (N-(2-hydroxyethyl)piperazine-N′-(2-ethanesulfonic acid) (HEPES), MOPS buffer (3-(N-morpholino)propanesulfonic acid), ACES (2-[(2-amino-2-oxoethyl)amino]ethanoesulfonic acid) buffer, ADA (N-(2-acetamido)2-iminodiacetic acid) buffer, AMPSO (3-[(1,1-dimethyl-2-hydroxyethyl)amino]-2-propanesulfonic acid) buffer, BES (N,N-bis(2-hydroxyethyl)-2 aminoethanesulfonic acid buffer, Bicine (N,N-bis(2-hydroxyethylglycine) buffer, Bis-Tris (bis-(2-hydroxyethyl)imino-tris(hydroxymethyl)methane buffer, CAPS (3-(cyclohexylamino)-1-propanesulfonic acid) buffer, CAPSO (3-(cyclohexylamino)-2-hydroxy-1-propanesulfonic acid) buffer, CHES (2-(N-cyclohexylamino)ethanesulfonic acid) buffer, DIPSO (3-[N,N-bis(2-hydroxyethyl)amino]-2-hydroxy-propanesulfonic acid) buffer, HEPPS(N-(2-hydroxyethylpiperazine)-N′-(3-propanesulfonic acid) buffer, HEPPSO(N-(2-hydroxyethyl)piperazine-N′-(2-hydroxypropanesulfonic acid) buffer, MES (2-(N-morpholino)ethanesulfonic acid) buffer, triethanolamine buffer, imidazole buffer, glycine buffer, ethanolamine buffer, phosphate buffer, MOPSO (3-(N-morpholino)-2-hydroxypropanesulfonic acid) buffer, PIPES (piperazine-N,N′-bis(2-ethanesulfonic acid) buffer, POPSO (piperazine-N,N′-bis(2-hydroxypropaneulfonic acid) buffer; TAPS(N-tris[hydroxymethyl)methyl-3-aminopropanesulfonic acid) buffer, TAPSO (3-[N-tris(hydroxymethyl)methylamino]-2-hydroxy-propanesulfonic acid) buffer, TES (N-tris(hydroxymethyl)methyl-2-aminoethanesulfonic acid) buffer, tricine (N-tris(hydroxymethyl)methylglycine buffer), 2-amino-2-methyl-1,3-propanediol buffer, 2-amino-2-methyl-1-propanol buffer, and combinations thereof.

7. A method according to claim 5 further comprising contacting the nucleic-acid containing test sample with at least one enzyme-inactivating component selected from the group consisting of manganese chloride, sodium lauroyl sarcosinate, and sodium dodecyl sulfate in the range of up to about 5% (w/v) concentration in the test sample.

8. A method according to claim 5 wherein the suppressant composition further comprises at least one nonionic detergent is selected from the group consisting of polyoxyethylene sorbitan monolaurates, octyl- and nonyl-phenoxypolyethoxylethanols (Nonidet detergents), octyl glucopyranosides, dodecyl maltopyranosides, heptyl thioglucopyranosides, Big CHAP detergents, Genapol X-80, Pluronic detergents, polyoxyethylene esters of alkylphenols (Triton), and derivatives and analogues thereof.

9. A test sample comprising:
(a) at least one nucleic acid
(b) a buffered solution comprising:
(i) a chelator selected from the group consisting of ethylenediaminetetraacetic acid (EDTA); imidazole; [ethylenebis(oxyethylenenitrilo)]tetraacetic acid (EGTA); iminodiacetate (IDA); 1,2-bis(2-aminophenoxy)ethane-N,N,N′,N′-tetraacetic acid (BAPTA); bis(5-amidino-2-benzimidazolyl)methane (BABIM) and salts thereof;
(ii) a chelator enhancing component selected from the group consisting of lithium chloride, sodium salicylate, sodium perchlorate, sodium thiocyanate, and combinations thereof; and
(iii) a buffer,
wherein the concentration of the chelator in the test sample is from about 0.2 M to about 0.6 M,
wherein the concentration of the chelator enhancing component in the test sample is from about 0.1 M to 0.9 M, and
wherein the pH of the test sample is from about 4.5 to about 8.0.

10. A test sample according to claim 9 wherein the nucleic acid comprises a nucleic acid selected from the group consisting of eukaryotic DNA, cDNA, RNA and combinations thereof.

11. A test sample according to claim 9, wherein the buffer is selected from the group consisting of potassium acetate, sodium acetate, potassium phosphate, sodium phosphate, tris(hydroxymethyl)aminomethane (Tris), (N-(2-hydroxyethyl)piperazine-N′-(2-ethanesulfonic acid) (HEPES), MOPS buffer (3-(N-morpholino)propanesulfonic acid), ACES (2-[(2-amino-2-oxoethyl)amino]ethanoesulfonic acid) buffer, ADA (N-(2-acetamido)2-iminodiacetic acid) buffer, AMPSO (3-[(1,1-dimethyl-2-hydroxyethyl)amino]-2-propanesulfonic acid) buffer, BES (N,N-bis(2-hydroxyethyl)-2 aminoethanesulfonic acid buffer, Bicine (N,N-bis(2-hydroxyethylglycine) buffer, Bis-Tris (bis-(2-hydroxyethyl)imino-tris(hydroxymethyl)methane buffer, CAPS (3-(cyclohexylamino)-1-propanesulfonic acid) buffer, CAPSO (3-(cyclohexylamino)-2-hydroxy-1-propanesulfonic acid) buffer, CHES (2-(N-cyclohexylamino)ethanesulfonic acid) buffer, DIPSO (3-[N,N-bis(2-hydroxyethyl)amino]-2-hydroxy-propanesulfonic acid) buffer, HEPPS(N-(2-hydroxyethylpiperazine)-N′-(3-propanesulfonic acid) buffer, HEPPSO(N-(2-hydroxyethyl)piperazine-N′-(2-hydroxypropanesulfonic acid) buffer, MES (2-(N-morpholino)ethanesulfonic acid) buffer, triethanolamine buffer, imidazole buffer, glycine buffer, ethanolamine buffer, phosphate buffer, MOPSO (3-(N-morpholino)-2-hydroxypropanesulfonic acid) buffer, PIPES (piperazine-N,N′-bis(2-ethanesulfonic acid) buffer, POPSO (piperazine-N,N′-bis(2-hydroxypropaneulfonic acid) buffer; TAPS(N-tris[hydroxymethyl)methyl-3-aminopropanesulfonic acid) buffer, TAPSO (3-[N-tris(hydroxymethyl)methylamino]-2-hydroxy-propanesulfonic acid) buffer, TES (N-tris(hydroxymethyl)methyl-2-aminoethanesulfonic acid) buffer, tricine (N-tris(hydroxymethyl)methylglycine buffer), 2-amino-2-methyl-1,3-propanediol buffer, 2-amino-2-methyl-1-propanol buffer, and combinations thereof.

12. A test sample according to claim 9, wherein the buffer solution further comprises at least one enzyme-inactivating component selected from the group consisting of manganese chloride, sodium lauroyl sarcosinate, and sodium dodecyl sulfate in the range of up to about 5% (w/v) concentration in the test sample.

13. A test sample according to claim 9 wherein the buffer solution further comprises at least one nonionic detergent is selected from the group consisting of polyoxyethylene sorbitan monolaurates, octyl- and nonyl-phenoxypolyethoxylethanols (Nonidet detergents), octyl glucopyranosides, dodecyl maltopyranosides, heptyl thioglucopyranosides, Big CHAP detergents, Genapol X-80, Pluronic detergents, polyoxyethylene esters of alkylphenols (Triton), and derivatives and analogues thereof.
